(19) United States
(12) Patent Application Publication
(10) Pub. No.: US 2026/0151382 A1
(43) Pub. Date: Jun. 4, 2026

(54) PRODUCTS OF MANUFACTURE AND METHODS FOR TREATING, PREVENTING, AMELIORATING OR PREVENTING CORONAVIRUS INFECTION

(71) Applicant: TOPELIA AUSTRALIA PTY LTD (ACN 652 771 650), Five Dock (AU)

(72) Inventor: Thomas Julius BORODY, Five Dock (AU)

(21) Appl. No.: 19/392,782

(22) Filed: Nov. 18, 2025

Related U.S. Application Data
(63) Continuation of application No. 17/371,009, filed on Jul. 8, 2021, now abandoned.
Application No. 17/371,009 is a division of application No. 16/828,891, filed on Mar. 24, 2020.
(60) Provisional application No. 62/971,803, filed on Feb. 7, 2020.
(60) Provisional application No. 62/972,486, filed on Feb. 10, 2020.
(60) Provisional application No. 62/988,852, filed on Mar. 12, 2020.
(60) Provisional application No. 62/990,283, filed on Mar. 16, 2020.
(60) Provisional application No. 62/992,137, filed on Mar. 19, 2020.

(51) Int. Cl. A61K 31/4706; A61K 9/00; A61K 31/137; A61K 31/215; A61K 31/426; A61K 31/436; A61K 31/4409; A61K 31/513; A61K 31/7052; A61K 38/21; A61K 39/215; A61K 39/395; A61M 15/00; A61M 15/08; A61P 31/14; C12N 7/00

(52) U.S. Cl. CPC A61K 31/4706; A61K 9/0019; A61K 9/0053; A61K 9/0075; A61K 9/0078; A61K 31/137; A61K 31/215; A61K 31/426; A61K 31/436; A61K 31/4409; A61K 31/513; A61K 31/7052; A61K 38/21; A61K 39/215; A61K 39/3955; A61M 15/0045; A61M 15/0068; A61M 15/009; A61M 15/08; A61P 31/14; C12N 7/00; A61M 2202/064; C12N 2770/20034

(57) ABSTRACT

Provided are pharmaceutical compositions comprising combinations of drugs, including products of manufacture and kits, and methods for using them, for treating, preventing, ameliorating, slowing the progress of, decreasing the severity of or preventing a coronavirus infection, or an infection caused by a virus in the subfamily Orthocoronavirinae, or a virus in the family Coronaviridae, or a virus in the order Nidovirales. In alternative embodiments, combinations, or cocktails, of a drug or drugs as provided herein are administered either enterally, parenterally and/or by inhalation. In alternative embodiments, combinations, or cocktails, of drugs as provided herein are used to block intracellular metabolic pathways and prevent progression of the infection to clinical illness and death. In alternative embodiments, novel aerosol or powder formulations for inhalation are provided.

### RELATED APPLICATIONS

This new United States Patent Application is a continuation of United States Patent Application Serial No. (USSN) Ser. No. 17/371,009, filed Jul. 8, 2021, now pending, which is a divisional of United States Patent Application Serial No. (USSN) Ser. No. 16/828,891, filed Mar. 24, 2020, which claims benefit of priority to U.S. provisional patent application Ser. No. 62/971,803, filed Feb. 7, 2020, and U.S. provisional patent application Ser. No. 62/972,486 filed Feb. 10, 2020, and U.S. provisional patent application Ser. No. 62/988,852 filed Mar. 12, 2020, and U.S. provisional patent application Ser. No. 62/990,283 filed Mar. 16, 2020, and U.S. provisional patent application Ser. No. 62/992,137 filed Mar. 19, 2020. The aforementioned applications are expressly incorporated herein by reference in their entirety and for all purposes.

### TECHNICAL FIELD

This invention generally relates to infectious diseases. In alternative embodiments, provided are pharmaceutical compositions comprising combinations of drugs, including products of manufacture and kits, and methods for using them, for treating, preventing, ameliorating, slowing the progress of, decreasing the severity of or preventing a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales. In alternative embodiments, combinations, or cocktails, of a drug or drugs as provided herein are administered either enterally, parenterally and/or by inhalation. In alternative embodiments, combinations, or cocktails, of drugs as provided herein are used to block intracellular metabolic pathways and prevent progression of the infection to clinical illness and death. In alternative embodiments, novel aerosol or powder formulations for inhalation are provided. In alternative embodiments, provided are therapeutic combinations of drugs or a drug, a pharmaceutical dosage form, a drug delivery device, or a product of manufacture, comprising: opaganib or YELIVA™, or opaganib or YELIVA™ and oral and/or inhaled chloroquine (or ARALEN™), chloroquine phosphate, chloroquine diphosphate and/or hydroxychloroquine (e.g., PLAQUENIL™), wherein optionally each or both of the opaganib and the chloroquine (or ARALEN™), chloroquine phosphate, chloroquine diphosphate and/or hydroxychloroquine (e.g., PLAQUENIL™) are in or formulated as a formulation for inhalation, for example, formulated as an aerosol, liquid or powder.

### BACKGROUND

Coronavirus infections have previously caused SARS (Severe Acute Respiratory Syndrome) and MERS (Middle East Respiratory Syndrome) and are particularly difficult to treat with anti-viral agents, and single drug regimens have not been found to be effective against the current coronavirus infection (2019-nCoV). The coronavirus infection (2019-nCoV), which started in China in December 2019, has spread rapidly throughout the world and has claimed hundreds of lives in China. The known coronavirus anti-infective agents used alone are unable to cure the infection.

### SUMMARY

In alternative embodiments, provided are therapeutic combinations of drugs or a drug, a pharmaceutical dosage form, a drug delivery device, or a product of manufacture, comprising:
(a) opaganib or YELIVA™, or opaganib or YELIVA™ and oral and/or inhaled chloroquine (or ARALEN™), chloroquine phosphate, chloroquine diphosphate and/or hydroxychloroquine (e.g., PLAQUENIL™), wherein optionally each or both of the opaganib and the chloroquine (or ARALEN™), chloroquine phosphate, chloroquine diphosphate and/or hydroxychloroquine (e.g., PLAQUENIL™) are in or formulated as a formulation for inhalation, for example, formulated as an aerosol, liquid or powder, or each or both are formulated for oral, intramuscular or intravenous administration, wherein optionally the opaganib is administered at a dosage of QD (once a day), bid (twice a day) or tid (three times a day) at a dosage of between about 100 to 600 mg per day or per dosage, or at about 100, 200, 300, 400, 500 or 600 mg per day or per dosage;
(b) lopinavir, ritonavir and oseltamivir (e.g., TAMIFLU™);
(c) lopinavir combined (formulated) with ritonavir, or KALETRA™, ALTERA™, ALUVIA™, KALMELTREX, LOPIMUNE™ or LOPINAVIR™, or lopinavir and ritonavir separately formulated;
(d) lopinavir combined (formulated) with ritonavir (or KALETRA™, ALTERA™, ALUVIA™, KALMELTREX, LOPIMUNE™ or LOPINAVIR™), or lopinavir and ritonavir, and oseltamivir (e.g., TAMIFLU™), optionally also with inhaled formulations or versions of chloroquine (or ARALEN™), chloroquine phosphate, chloroquine diphosphate and/or hydroxychloroquine (e.g., PLAQUENIL™) and/or oral chloroquine (or ARALEN™), chloroquine phosphate, chloroquine diphosphate and/or hydroxychloroquine (e.g., PLAQUENIL™) simultaneously;
(e) lopinavir, ritonavir, chloroquine and oseltamivir (or TAMIFLU™); wherein optionally the chloroquine comprises inhaled chloroquine (or ARALEN™), chloroquine phosphate, chloroquine diphosphate and/or hydroxychloroquine (e.g., PLAQUENIL™) and/or oral chloroquine (or ARALEN™), chloroquine phosphate, chloroquine diphosphate and/or hydroxychloroquine (e.g., PLAQUENIL™) simultaneously;
(f) lopinavir and oseltamivir (e.g., TAMIFLU™);
(g) ritonavir and oseltamivir (e.g., TAMIFLU™);
(h) remdesivir (e.g., GS-5734™, Gilead Sciences) alone, or oseltamivir (e.g., TAMIFLU™) and remdesivir (e.g., GS-5734™, Gilead Sciences), and optionally the remdesivir is an oral formulation and/or an inhaled remdesivir formulation;
(i) oseltamivir (e.g., TAMIFLU™) and efavirenz (e.g., SUSTIVA™);
(j) oseltamivir (e.g., TAMIFLU™) and nevirapine (or the combination efavirenz with emtricitabine and tenofovir, or ATRIPLA™);
(k) oseltamivir (or TAMIFLU™) and amprenavir (e.g., AGENERASE™);
(l) oseltamivir (e.g., TAMIFLU™) and nelfinavir (e.g., VIRACEPT™); or
(m) a thiazolide class drug, optionally nitazoxanide (or ALINIA™, NIZONIDE™) or tizoxanide (or 2-Hydroxy-N-(5-nitro-2-thiazolyl)benzamide), with or in combination with any of (a) to (i), or the thiazolide class drug (e.g., nitazoxanide or tizoxanide) and oseltamivir (or TAMIFLU™);
(n) plitidepsin (also known as dehydrodidemnin B), or APLIDIN™ (PharmaMar, S.A.);
(o) an inhibitor or S-phase kinase-associated protein 2(SKP 2 ), or dioscin, or niclosamide, or NICLOCIDE™, FENASAL™, or PHENASAL™;
(p) ribavirin, interferon beta 1b, or a combination of ribavirin and interferon beta, or a combination of lopinavir and ritonavir and interferon-beta-lb;
(q) abacavir, actemra, acyclovir e.g., (ACICLOVIR™), adefovir, amantadine, ampligen, amprenavir (e.g., AGENERASE™), aprepitant, arbidol, atazanavir, atripla, balavir, baloxavir marboxil (XOFLUZA™), bepotastine, bevirimat, bictegravir, biktarvy, brilacidin, cidofovir, caspofungin, lamivudine and zidovudine (e.g., COMBVIR™) , cobicstat, colisitin, cocaine, darunavir, delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II and/or interferon type III), lamivudine, lopinavir, loviride, ledipasvir, leronlimab, maraviroc, methisazone, moroxydine, nelfinavir, nevirapine, nexavir, nitazoxanide, norvir, a nucleoside analogue (optionally brincidofovir, didanosine, favipiravir (also known as T-705, avigan, or favilavir, Toyama Chemical, Fujifilm, Japan), vidarabine, galidesivir (e.g., BCX4430, IMMUCILLIN-A™), remdesivir (e.g., GS-5734™, Gilead Sciences), cytarabine, gemcitabine, emtricitabine, lamivudine, zalcitabine, abacavir, acyclovir, entecavir, stavudine, telbivudine, zidovudine, idoxuridine and/or trifluridine or any combination thereof), oseltamivir (or TAMIFLU™), peginterferon alfa-2a, penciclovir, peramivir (e.g., RAPIVAB™), perfenazine, pleconaril, plurifloxacin, podophyllotoxin, pyramidine, raltegravir, rifampicin, ribavirin, rilpivirine, rimantadine, ritonavir, saquinavir, sofosbuvir, stavudine, telaprevir, tegobuv, tenofovir alafenamide, tenofovir disoproxil, tenofovir, tipranavir, trifluridine, trizivir, tromantadine, truvada, valaciclovir (e.g., VALTREX™), valganciclovir, valrubicin, vapreotide, vicriviroc, vidarabine, viramidine, velpatasvir, vivecon, zalcitabine, zanamivir (e.g., RELENZA™), zidovudine or any combination thereof;
(r) an mucolytic therapy or drug, optionally acetylcysteine, ambroxol, bromhexine, carbocisteine, erdosteine, mecysteine or domase alfa, or an expectorant, optionally guaifenesin;
(s) a viral, or a coronavirus or a COVID-19, protease inhibitor, optionally ASC09 (CAS registry no. 1000287-05-7) (Janssen Research and Development, LLC), ritonavir or ASC09 and ritonavir, or a JAK1/2 inhibitor (optionally baricitinib), optionally compound 11r (University of Lubeck, Germany, see e.g., Zhang et al J. Med Chem 2020, Feb. 11, 2020), or darunavir, cobicistat or darunavir and cobicistat;
(t) an angiotensin-converting enzyme 2(ACE 2 ) inhibitor, optionally to block the site of viral spike protein interaction for anti-SARS-CoV-2 infection control;
(u) an anti-vascular endothelial growth factor (VEGF) (optionally VEGF-A) drug or antibody, optionally bevacizumab;
(v) a serine protease inhibitor, optionally camostat;
(w) anti-PD-1 checkpoint inhibitor, optionally camrelizumab;
(x) a compound or antibody capable of binding complement factor CS and blocking membrane attack complex formation, optionally eculizumab;
(y) a cathepsin inhibitor, optionally a cathepsin K, B or L inhibitor, optionally relacatib;
(z) thalidomide, or thalidomide and glucocorticoid (optionally low-dose glucocorticoid), or and thalidomide and celecoxib;
(aa) an antibacterial antibiotic or a macrolide drug, wherein optionally the macrolide drug comprises azithromycin (e.g., ZITHROMAX™, or AZITHROCIN™), clarithromycin (e.g., BIAXIN™), erythromycin (e.g., ERYTHROCIN™), or fidaxomicin (e.g., DIFICID™ or DIFICLIR™), troleandomycin (e.g., TEKMISIN™), tylosin (e.g., TYLOCINE™ or TYLAN™), solithromycin (e.g., SOLITHERA™), oleandomycin (or SIGMAMYCINE™), midecamycin, roxithromycin, kitasamycin or turimycin, josamycin, carbomycin or magnamycin, and/or spiramycin;
(bb) chloroquine (or ARALEN™), chloroquine phosphate, chloroquine diphosphate and/or hydroxychloroquine (e.g., PLAQUENIL™) with (or formulated with) or in combination with any of (a) to (aa), or chloroquine, chloroquine phosphate, chloroquine diphosphate and/or hydroxychloroquine (e.g., PLAQUENIL™) and oseltamivir (or TAMIFLU™);
(cc) chloroquine (e.g., ARALEN™), chloroquine phosphate, chloroquine diphosphate and/or
hydroxychloroquine (e.g., PLAQUENIL™);
(dd) any one or several or all of (a) to (cc) with (or formulated with or formulated as an) inhaled formulation and/or formulated with or formulated as an oral, intramuscular (IM) or intravenous (IV) formulation, wherein optionally both the inhaled and the oral, IV and/or IM formulations are administered simultaneously or sequentially, and optionally the inhaled formulation comprises chloroquine (or ARALEN™), chloroquine phosphate, chloroquine diphosphate and/or hydroxychloroquine (e.g., PLAQUENIL™) and/or oral chloroquine (or ARALEN™), chloroquine phosphate, chloroquine diphosphate and/or hydroxychloroquine (e.g., PLAQUENIL™) administered simultaneously or overlapping; or
(ee) any combination of (a) to (dd).

In alternative embodiments of the therapeutic combinations of the drugs or drug, pharmaceutical dosage form, drug delivery device, or product of manufacture as provided herein:
the drugs or drug, pharmaceutical dosage form, drug delivery device, or product of manufacture further comprise (e.g., are formulated with, or are administered with) an (or an additional) anti-viral drug or medication, or anti microbial drug, or palliative agent or drug,
   wherein optionally the anti-viral drug or medication, or anti-microbial drug, is or comprises efavirenz (e.g., SUSTIVA™), tenofovir, emtricitabine and tenofovir, nevirapine (or the combination efavirenz with emtricitabine and tenofovir, or ATRIPLA™), amprenavir (e.g., AGENERASE™), nelfinavir (e.g., VIRACEPT™) and/or remdesivir (e.g., GS-5734™, Gilead Sciences), a viral RNA-dependent RNA polymerase inhibitor, optionally galidesivir,
   and optionally the anti-viral drug or medication is or comprises an anti-retroviral drug or drug combination, and optionally the anti-retroviral drug or drug combination comprises: darunavir and cobicistat (e.g., REZOLSTA™ or PREZCOBIX™); atazanavir and cobicistat (or EVOTAZ™); abacavir, lamivudine and dolutegravir (TRIUMEQ™); tenofovir (or disoproxil or emtricitabine) and elvitegravir and cobicistat (e.g., STRIBILD™); tenofovir (or disoproxil or emtricitabine) and elvitegravir and cobicistat (COMPLERA™ or EVIPLERA™); efavirenz, emtricitabine and tenofovir (ATRIPLA); lamivudine, nevirapine and stavudine (e.g., TRIOMUNE™); atazanavir and cobicistat (e.g., EVOTAZ™); lamivudine and raltegravir (e.g., DUTREBIS™); lamivudine and dolutegravir (or DOVATO™); doravirine, lamivudine and tenofovir (e.g., DELSTRIGO™); or lamivudine, zidovudine and nevirapine (e.g., CUOVIR-N™);
   and optionally the additional anti-viral drug or medication, or anti-microbial drug, is formulated with the chloroquine (e.g., ARALEN™), chloroquine phosphate, chloroquine diphosphate, hydroxychloroquine (e.g., PLAQUENIL™), lopinavir, ritonavir and/or oseltamivir or is formulated separately from the chloroquine (e.g., ARALEN™), chloroquine phosphate, chloroquine diphosphate, hydroxychloroquine (e.g., PLAQUENIL™), lopinavir, ritonavir and/or oseltamivir,
   and optionally the anti-viral drug or medication, or anti-microbial drug, or palliative agent comprises or further comprises zinc or vitamin C, which optionally is given enterally or parenterally;
the chloroquine (e.g., ARALEN™), chloroquine phosphate, chloroquine diphosphate, hydroxychloroquine (e.g., PLAQUENIL™), lopinavir, ritonavir and/or oseltamivir are formulated separately or together, or the lopinavir and ritonavir are formulated together and the oseltamivir is formulated separately;
the therapeutic combination of drugs or drug, pharmaceutical dosage form, or the chloroquine (e.g., ARALEN™), chloroquine phosphate, chloroquine diphosphate, hydroxychloroquine (e.g., PLAQUENIL™), lopinavir, ritonavir and/or oseltamivir and/or the anti-viral drug or medication, or anti-microbial drug are formulated or contained in a liquid formulation (optionally sterile saline or water), a spray, a powder, an aerosol, or any formulation for inhalation, a pill, a capsule, a tablet, or a geltab, or equivalents;
and optionally the therapeutic combination of drugs or drug, pharmaceutical dosage form, or the chloroquine (e.g., ARALEN™), chloroquine phosphate, chloroquine diphosphate, hydroxychloroquine (e.g., PLAQUENIL™), lopinavir, ritonavir and/or oseltamivir and/or the anti-viral drug or medication, or anti-microbial drug, are coated on the surface of or contained in: a bead, a powder, a particle, or a multilayered bead or particle, and optionally the bead, powder, particle or the multilayered bead or particle is contained in a pill, a capsule, a tablet, or a geltab, or equivalents, for oral delivery, wherein optionally the pill, capsule, tablet, geltab or equivalent for oral delivery is a hard gelatin capsule or equivalent, or comprises a hard gelatin or equivalent; and/or
the lopinavir, ritonavir and oseltamivir are formulated or packaged for administration as or dosing in the ratio of 25:100:75 of lopinavir: ritonavir: oseltamivir.

In alternative embodiments, provided are drug delivery devices or packages, a kit, a blister package, a clamshell or a tray, comprising a therapeutic combination of drugs or drug, a pharmaceutical dosage form or a formulation of any of the preceding claims, wherein the drug delivery device comprises an inhalation device or inhaler or a nasal spray device, and optionally the inhaler or a nasal spray device is a hand-held inhaler or a nasal spray device, and optionally the inhaler or a nasal spray device is a metered or dose-counting inhaler or a nasal spray device,
wherein the drug delivery device or package, blister pack, clamshell or tray comprises a plurality of compartments spatially arranged on the drug delivery device or package, blister pack, clamshell or tray to follow a dosage administration regimen, wherein the spatially arranged plurality of compartments are in at least two rows, each row marked for the time for which a drug of the drug combination, or the tablets, pills, capsules, geltabs or equivalents, are to be taken by a user (optionally a patient), optionally one row marked for morning, breakfast or AM administration, and one row marked for evening, dinnertime or PM administration, and optionally the row or rows marked for morning, breakfast or AM administration is or are positioned above the row or rows marked for evening, dinnertime or PM administration,
and optionally the spatially arranged plurality of compartments are in four rows, two rows marked for morning, breakfast or AM administration, and two rows marked for evening, dinnertime or PM administration,
and optionally the spatially arranged plurality of compartments are arranged to facilitate and/or direct the patient to take the drugs in that row two times a day (bid), three times a day (tid), four times a day, or up to ten times a day (wherein optionally the higher amounts are for the very sick), optionally also comprising indication of actual times for patient to ingest the drugs in each row, and if appropriate, directions to also ingest fluids or other drugs and/or food (e.g., small amounts of food),
and optionally each row comprises seven compartments for one dosage administration for each day of the week, or eight compartments for one dosage administration for each day of the week and one spare, and optionally each vertically arranged set of compartments, or columns, are marked for which day of the week the dosage formulations contained therein are to be taken by the user, and optionally where the drug delivery device or package, blister pack, clamshell or tray has one row for morning, breakfast or AM administration, and one row marked for evening, dinnertime or PM administration, each column or day will have two compartments, optionally where the compartment for morning, breakfast or AM administration is above the compartment for evening, dinnertime or PM administration,
and optionally each compartment has a foil or equivalent backing, or each compartment is an environmentally- (optionally moisture-, pathogen-, and/or light-) protected or sealed storage unit, and optionally the foil backing requires minimal finger strength to remove a dosage formulation (optionally one, two or three or more capsules, tablets, pills, geltabs or equivalents) in the compartment,
and optionally the blister package is a face seal blister package, a gang run blister package, a mock blister package, an interactive blister package or a slide blister package,
and optionally the drug delivery device or package, blister package, clamshell or tray is joined with board material which allows the product to be packaged, handled, hung, displayed and/or shipped without damaging the blister protection or seal, and optionally also provided with child resistant features,
and optionally the drug delivery device or package, blister package, clamshell or tray comprises a medical electronic monitory system that records administration time and transmits information to near-field communication (NFC) enabled mobile phone,
wherein optionally the kit is a travel kit comprising instructions of use by the traveler, wherein the instructions optionally comprise instructing the traveler to immediately take an indicated dosage, optionally a first arrange row of drugs on the drug delivery device or package, blister package, clamshell or tray, if they believe they have been in contact with or in near contact with or exposed to an individual that is infect or might be infected with coronavirus (e.g., a COVID-19) or to an individual having fever, sore throat, rigors or shakes, chest pain on breathing, coughing, diarrhea and/or muscle aches.

In alternative embodiments, provided are methods for treating, preventing, ameliorating, slowing the progress of, decreasing the severity of or preventing a coronavirus infection, or a COVID-19 or a 2019-nCoV (or so-called Wuhan coronavirus) infection, or an infection caused by a virus in the sub-family Orthocoronavirinae, or a virus in the family Coronaviridae, or a virus in the order Nidovirales, comprising administered to an individual in need thereof a therapeutic combination of drugs or drug, a pharmaceutical dosage form, a drug delivery device, or a product of manufacture as provided herein, wherein optionally the therapeutic combinations of drugs or drugs or pharmaceutical dosage forms are administered as inhaled formulations (e.g., powders, liquids, aerosols) and/or are administered (optionally simultaneously, or sequentially) with oral, intravenous (IV) or intramuscular formulations.

In alternative embodiments of the methods, the administered therapeutic combination of drugs or drug, pharmaceutical dosage form, drug delivery device, or product of manufacture comprises, or the method of administration comprises:
(a) opaganib or YELIVA™, or opaganib or YELIVA™ and oral and/or inhaled chloroquine (or ARALEN™), chloroquine phosphate, chloroquine diphosphate and/or hydroxychloroquine (e.g., PLAQUENIL™) wherein optionally each or both of the opaganib and the chloroquine (or ARALEN™), chloroquine phosphate, chloroquine diphosphate and/or hydroxychloroquine (e.g., PLAQUENIL™) are in or formulated as a formulation for inhalation, for example, formulated as an aerosol, liquid or powder, or each or both are formulated for oral, intramuscular or intravenous administration, and optionally each are simultaneously administered in both oral and in inhalation forms, or only is administered as an inhalant;
(b) lopinavir, ritonavir and oseltamivir (e.g., TAMIFLU™);
(c) lopinavir combined (formulated) with ritonavir, or KALETRA™, ALTERA™, ALUVIA™, KALMELTREX, LOPIMUNE™ or LOPINAVIR™, or lopinavir and ritonavir separately formulated;
(d) lopinavir combined (formulated) with ritonavir, or KALETRA™, ALTERA™, ALUVIA™, KALMELTREX, LOPIMUNE™ or LOPINAVIR™, and oseltamivir (e.g., TAMIFLU™), optionally also with inhaled chloroquine (or ARALEN™), chloroquine phosphate, chloroquine diphosphate and/or hydroxychloroquine (e.g., PLAQUENIL™) and/or oral chloroquine (or ARALEN™), chloroquine phosphate, chloroquine diphosphate and/or hydroxychloroquine (e.g., PLAQUENIL ™) simultaneously,
wherein optionally the dosage administration comprises: lopinavir about 200 mg twice daily, ritonavir about 50 mg twice daily, chloroquine about 250 mg twice daily, oseltamivir (TAMIFLU™) about 75 mg twice daily;
(e) lopinavir, ritonavir and oseltamivir (or TAMIFLU™); with inhaled chloroquine (or ARALEN™), chloroquine phosphate, chloroquine diphosphate and/or hydroxychloroquine (e.g., PLAQUENIL™) and/or oral chloroquine (or ARALEN™), chloroquine phosphate, chloroquine diphosphate and/or hydroxychloroquine (e.g., PLAQUENIL™) simultaneously;
(f) lopinavir and oseltamivir (e.g., TAMIFLU™);
(g) ritonavir and oseltamivir (e.g., TAMIFLU™);
(h) remdesivir (e.g., GS-5734™, Gilead Sciences) alone, or oseltamivir (e.g., TAMIFLU™) and remdesivir (e.g., GS-5734™, Gilead Sciences);
(i) oseltamivir (e.g., TAMIFLU™) and efavirenz (e.g., SUSTIVA™);
(j) oseltamivir (e.g., TAMIFLU™) and nevirapine (or the combination efavirenz with emtricitabine and tenofovir, or ATRIPLA™);
(k) oseltamivir (or TAMIFLU™) and amprenavir (e.g., AGENERASE™);
(l) oseltamivir (e.g., TAMIFLU™) and nelfinavir (e.g., VIRACEPT™); or
(m) a thiazolide class drug, optionally nitazoxanide (or ALINIA™, NIZONIDE™) or tizoxanide (or 2-Hydroxy-N-(5-nitro-2-thiazolyl)benzamide), with or in combination with any of (a) to (i), or the thiazolide class drug (e.g., nitazoxanide or tizoxanide) and oseltamivir (or TAMIFLU™);
(n) plitidepsin (also known as dehydrodidemnin B), or APLIDIN™ (PharmaMar, S.A.);
(o) an inhibitor or S-phase kinase-associated protein 2(SKP 2 ), or dioscin, or niclosamide, or NICLOCIDE™, FENASAL™, or PHENASAL™;
(p) ribavirin, interferon beta 1b, or interferon alfa-2b, or a combination of ribavirin and an interferon, for example, interferon alpha or beta (e.g., interferon beta 1b or interferon alfa- 2b), or a combination of lopinavir and ritonavir and an interferon, e.g., interferon-beta-lb and/or interferon alfa-2b;
(q) abacavir, actemra, acyclovir e.g., (ACICLOVIR™), adefovir, amantadine, ampligen, amprenavir (e.g., AGENERASE™), aprepitant, arbidol, atazanavir, atripla, balavir, baloxavir marboxil (XOFLUZA™), bepotastine, bevirimat, bictegravir, biktarvy, brilacidin, cidofovir, caspofungin, lamivudine and zidovudine (e.g., COMBVIR™), cobicstat, colisitin, cocaine, darunavir, delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, favilavir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II, interferon alfa-2b, and/or interferon type III), lamivudine, lopinavir, loviride, ledipasvir, leronlimab, maraviroc, methisazone, moroxydine, nelfinavir, nevirapine, nexavir, nitazoxanide, norvir, a nucleoside analogue (optionally didanosine, vidarabine, galidesivir (e.g., BCX4430, IMMUCILLIN-A™), remdesivir (e.g., GS-5734™, Gilead Sciences), cytarabine, gemcitabine, emtricitabine, lamivudine, zalcitabine, abacavir, acyclovir, entecavir, stavudine, telbivudine, zidovudine, idoxuridine and/or trifluridine or any combination thereof), oseltamivir (or TAMIFLU™), peginterferon alfa-2a or alfa-2b, penciclovir, peramivir (e.g., RAPIVAB™), perfenazine, pleconaril, plurifloxacin, podophyllotoxin, pyramidine, raltegravir, rifampicin, ribavirin, rilpivirine, rimantadine, ritonavir, saquinavir, sofosbuvir, stavudine, telaprevir, tegobuv, tenofovir alafenamide, tenofovir disoproxil, tenofovir, tipranavir, trifluridine, trizivir, tromantadine, truvada, valaciclovir (e.g., VALTREX™), valganciclovir, valrubicin, vapreotide, vicriviroc, vidarabine, viramidine, velpatasvir, vivecon, zalcitabine, zanamivir (e.g., RELENZA™), zidovudine or any combination thereof; (q) chloroquine (or ARALEN™), chloroquine phosphate, chloroquine diphosphate and/or hydroxychloroquine (e.g., PLAQUENIL™) with or in combination with any of (a) to (p), or chloroquine chloroquine phosphate, chloroquine diphosphate and/or hydroxychloroquine (e.g., PLAQUENIL™) and oseltamivir (or TAMIFLU™);
(r) an mucolytic therapy or drug, optionally acetylcysteine, ambroxol, bromhexine, carbocisteine, erdosteine, mecysteine or domase alfa, or an expectorant, optionally guaifenesin;
(s) a viral, or a coronavirus or a COVID-19, protease inhibitor, optionally ASC09 (CAS registry no. 1000287-05-7) (Janssen Research and Development, LLC), ritonavir or ASC09 and ritonavir, or a JAK1/2 inhibitor (optionally baricitinib), optionally compound 11r (University of Lubeck, Germany, see e.g., Zhang et al J. Med Chem 2020, Feb. 11, 2020), or darunavir, cobicistat or darunavir and cobicistat;
(t) an angiotensin-converting enzyme 2(ACE 2 ) inhibitor, optionally to block the site of viral spike protein interaction for anti-SARS-CoV-2 infection control;
(u) an anti-vascular endothelial growth factor (VEGF) (optionally VEGF-A) drug or antibody, optionally bevacizumab;
(v) a serine protease inhibitor, optionally camostat;
(w) anti-PD-1 checkpoint inhibitor, optionally camrelizumab;
(x) a compound or antibody capable of binding complement factor C5 and blocking membrane attack complex formation, optionally eculizumab;
(y) a cathepsin inhibitor, optionally a cathepsin K, B or L inhibitor, optionally relacatib;
(z) thalidomide, or thalidomide and glucocorticoid (optionally low-dose glucocorticoid), or and thalidomide and celecoxib;
(aa) an antibacterial antibiotic or a macrolide drug, wherein optionally the macrolide drug comprises azithromycin (e.g., ZITHROMAX™, or AZITHROCIN™), clarithromycin (e.g., BIAXIN™), erythromycin (e.g., ERYTHROCIN™), or fidaxomicin (e.g., DIFICID™ or DIFICLIR™), troleandomycin (e.g., TEKMISIN™), tylosin (e.g., TYLOCINE™ or TYLAN™), solithromycin (e.g., SOLITHERA™), oleandomycin (or SIGMAMYCINE™), midecamycin, roxithromycin, kitasamycin or turimycin, josamycin, carbomycin or magnamycin, and/or spiramycin, and optionally the macrolide drug is administered at a dosage of about 100 to 200 mg a day, or at about 25 to 100 mg three times a day (tid), or at 50 mg tid;
(bb) chloroquine (e.g., ARALEN™), chloroquine phosphate, chloroquine diphosphate and/or hydroxychloroquine (e.g., PLAQUENIL™);
(cc) any one or several or all of (a) to (bb), or any therapeutic combination of drugs or drug, pharmaceutical dosage form of any of the preceding claims, are administered as an inhaled aerosol, powder or liquid or other inhalation formulation, or are administered with inhaled (e.g., aerosol or power administered by inhaler) chloroquine (or ARALEN™), chloroquine phosphate, chloroquine diphosphate and/or hydroxychloroquine (e.g., PLAQUENIL™),
and optionally the chloroquine, chloroquine phosphate, chloroquine diphosphate and/or hydroxychloroquine administration is started (early, first) 1, 2, 3, 4, 5 or 6 days before administration of the one or several of the drugs of (a) to (bb), wherein optionally this initial (early, first) administration of chloroquine, chloroquine phosphate, chloroquine diphosphate and/or hydroxychloroquine administration is by inhalation and/or by an oral, IM or IV formulation,
and optionally the chloroquine, chloroquine phosphate, chloroquine diphosphate and/or hydroxychloroquine administration is started with a high dose (e.g., a so-called “loading dose”), for example, an oral (e.g., a single dose such as a single tablet, capsule or pill), IV or IM dosage of between about 250 mg, 300 mg, 350 mg, 300 mg or 0.5 gram (gm) (500 mg) and 1.5 gm, or between about 400 mg and 1 gm, optionally with follow up administrations every 4 to 10 days, or every 1, 2, 3, 4, 5, 6, 7, 8, 9 or 10 days, at a lower dosage of between about 50 gm to 200 mg, or about 100 mg, total daily dosage, optionally continuing for between about one week to one month,
wherein optionally the initial (early, first) administration of the chloroquine, chloroquine phosphate, chloroquine diphosphate and/or hydroxychloroquine also comprises early, first administration of the macrolide drug, optionally azithromycin, and optionally the macrolide drug is started with a high dose (e.g., a so-called “loading dose”), optionally an oral (e.g., a single dose such as a single tablet, capsule or pill), IV or IM dosage of between about 400 mg to 0.5 gram (gm) (500 mg) and 1 gm, or at about 500 mg, optionally with follow up administrations every 4 to 10 days, or every 1, 2, 3, 4, 5, 6, 7, 8, 9 or 10 days, at a lower dosage of between about 100 gm to 300 mg, or about 250 mg, total daily dosage, optionally continuing for between about one week to one month,
wherein optionally the initial (early, first) administration of the chloroquine, chloroquine phosphate, chloroquine diphosphate and/or hydroxychloroquine and/or the macrolide drug also comprises early, first administration of opaganib or YELIVA™, wherein optionally the opaganib is administered at a dosage of QD (once a day), bid (twice a day) or tid (three times a day) at a dosage of between about 100 to 600 mg per day or per dosage, or at about 100, 200, 300, 400, 500 or 600 mg per day or per dosage,
and/or the any one or several or all of (a) to (cc), or any therapeutic combination of drugs or drug, pharmaceutical dosage form of any of the preceding claims, are administered orally, intramuscularly, subcutaneously, topically, by enema, intravaginally, or intravenously,
and optionally are administered with oral (e.g., pills, tablets, capsules, liquids) chloroquine (or ARALEN™), chloroquine phosphate, chloroquine diphosphate and/or hydroxychloroquine (e.g., PLAQUENIL™),
wherein the inhaled (e.g., aerosol or power administered by inhaler) therapeutic combination of drugs or drug, pharmaceutical dosage form, drug delivery device, or product of manufacture is or are administered simultaneously or overlapping or sequentially; or
(dd) any combination of (a) to (cc) simultaneously administered in both inhaled (e.g., aerosol or power administered by inhaler) and oral (e.g., pills, tablets, capsules, liquids), intramuscular, subcutaneous (SC) and/or intravenous (IV) forms or formulations; or
(ee) any combination of (a) to (dd).

In alternative embodiments of the methods:
the lopinavir, ritonavir and oseltamivir are formulated or packaged for administration as or dosing in the ratio of 25:100:75 of lopinavir: ritonavir: oseltamivir;
the chloroquine (e.g., ARALEN™), chloroquine phosphate, chloroquine diphosphate, hydroxychloroquine (e.g., PLAQUENIL™), lopinavir, ritonavir and/or oseltamivir, and/or the anti-viral drug or medication, or anti-microbial drug, are administered enterally or parenterally, or are administered orally, intramuscularly (IM), intravenously (IV), by inhalation, or by inhalation when formulated as an aerosol, a spray, a microparticle, a nanoparticle, or a powder;
the chloroquine (e.g., ARALEN™), chloroquine phosphate, chloroquine diphosphate, hydroxychloroquine (e.g., PLAQUENIL™), lopinavir, ritonavir and/or oseltamivir, and/or the anti-viral drug or medication, or anti-microbial drug, are administered one to ten times per day (e.g., bid, tid, or 5, 6, 7, 8, 9, or 10 or more times a day) depending on the stage of the condition (for example, exposed to infection, positivity in blood but asymptomatic, or symptomatic, mild or severe;
the method comprises first administering (alone) for about 7 days (or 2, 3, 4, 5, 6 or 7 or more days) oseltamivir (or TAMIFLU™) at 3 times per day (tid) (or alternatively twice a day (bid) or four times a day or more), followed by a blood sample to be taken for virus testing; and if and when virus blood positivity is confirmed, or viral infection is otherwise confirmed, the osteltamivir is supplemented by the lopinavir and ritonavir (which can be administered together or separately) three times daily (tid) (or alternatively twice a day (bid) or four times a day or more), while continuing the osteltamivir, for example, all three medications three (or two or four or more) times daily or 9 (or more) medications daily;
the duration of the combined drug therapy, or the chloroquine (e.g., ARALEN™), chloroquine phosphate, chloroquine diphosphate or hydroxychloroquine (e.g., PLAQUENIL™) therapy, is about 2, 3, 4, 5, 6, 7, 8, 9, 10, 11, 12, 13, 14, 15, 16, 17, 18, 19, 20 or 21 or more or more days or for as long as the patient tests positive for the coronavirus (e.g., a COVID-19) infection, or for at least 2, 3, 4, 5, 6, 7, 8, 9, 10, 11, 12, 13, 14, 15, 16, 17, 18, 19, 20 or 21 or more or more days after the patient no longer tests positive for coronavirus (e.g., a COVID-19), or wherein the combined drug therapy, or
   the chloroquine (e.g., ARALEN™), chloroquine phosphate, chloroquine diphosphate or hydroxychloroquine (e.g., PLAQUENIL™) therapy, begins (commences) as soon as an individual (a patient) learns (understands) he or she was in close proximity to a coronavirus (e.g., a COVID-19) positive individual, or believes that he or she may have been in close proximity to a coronavirus (e.g., a COVID-19) positive individual, or begins (commences) as soon as the individual (the patient) test positive for coronavirus (e.g., a COVID-19), or
   the chloroquine (e.g., ARALEN™), chloroquine phosphate, chloroquine diphosphate or hydroxychloroquine (e.g., PLAQUENIL™) therapy, continues until at least one, or at least two, tests show the individual (the patient) is negative for coronavirus (e.g., a COVID-19), or continues until all body fluids or samples (optionally respiratory or oral swabs or fluids, sputum, serology or blood or serum, and stool or fecal samples) test negative for coronavirus (e.g., a COVID-19),
   and optionally the individual (the patient) is retested at least every week, biweek or month to test or reinfection or reemergence or reoccurrence of viral infection or activity, and if the individual (the patient) again tests positive, then reinitiate treatment as used when the individual was first diagnosed with coronavirus (e.g., a COVID-19) infection;
the combined drug therapy, or the chloroquine (e.g., ARALEN™), chloroquine phosphate, chloroquine diphosphate or hydroxychloroquine (e.g., PLAQUENIL™) therapy, comprises administering the chloroquine (e.g., ARALEN™), chloroquine phosphate, chloroquine diphosphate or hydroxychloroquine (e.g., PLAQUENIL™):
   (a) until, or increasing the dosage of the chloroquine (e.g., ARALEN™), chloroquine phosphate, chloroquine diphosphate or hydroxychloroquine (e.g., PLAQUENIL™), until a steady state chloroquine (e.g., ARALEN™), chloroquine phosphate, chloroquine diphosphate or hydroxychloroquine (e.g., PLAQUENIL™) plasma concentration of at least 1 μg/mL (or at least approximately 3.125 μM/L) is achieved, or until a whole blood concentration of at least about 16 μM/L is achieved, and optionally sustained, until the patient's viremia becomes undetectable; or
   (b) intravenously (IV) at a dosage of between about 10 mg/kg to 20 mg/kg, or at about 15 mg/kg, over a one to 6 hour period, or over four hour period, and optionally the IV infusion is repeated, optionally every 6 to 12 hours, optionally for between about 2 to 20 repeat infusions;
the combined drug therapy, or the chloroquine (e.g., ARALEN™), chloroquine phosphate, chloroquine diphosphate or hydroxychloroquine (e.g., PLAQUENIL™) therapy, is formulated with or is administered before, during or after: an anti-coronavirus vaccine or a vaccination with an anti-coronavirus (e.g., an anti-COVID-19) vaccine, or administration of a passive immunity therapy (e.g., infusion (e.g., intravenous infusion, e.g., infusion of a hyperimmune globulin) of one or more antibodies capable of specifically binding to or neutralizing or stopping the multiplication or spread of a coronavirus (e.g., a COVID-19);
the combined drug therapy, or the chloroquine (e.g., ARALEN™), chloroquine phosphate, chloroquine diphosphate or hydroxychloroquine (e.g., PLAQUENIL™) therapy, is administered with (optionally before, during and/or after), or formulated with, an anti-inflammatory therapy or at least one antiinflammatory therapy drug, wherein optionally the anti-inflammatory therapy or drug comprises: a sphingosine kinase-2 (SK2) selective inhibitor (e.g., opaganib (e.g., YELIVA™), sirolimus, a JAK1/2 /TYK2 inhibitor (optionally ruxolitinib), an anti CD47 mAb (optionally meplazumab), COX (e.g., COX2) inhibitor, a glucocorticoid and/or an immunosuppressive agent, and optionally the immunosuppressive agent comprises tocilizumab or fingolimod; and/or
the combined drug therapy, or the chloroquine (e.g., ARALEN™), chloroquine phosphate, chloroquine diphosphate or hydroxychloroquine (e.g., PLAQUENIL™) therapy, is formulated with or is administered with (optionally before, during and/or after) administration of:
   (a) an mucolytic therapy or drug, optionally acetylcysteine, ambroxol, bromhexine, carbocisteine, erdosteine, mecysteine or domase alfa, or an expectorant, optionally guaifenesin;
   (b) a viral, or a coronavirus or a COVID-19, protease inhibitor, optionally ASC09 (CAS registry no. 1000287-05-7) (Janssen Research and Development, LLC), ritonavir or ASC09 and ritonavir, or a JAK1/2 inhibitor (optionally baricitinib), optionally compound 11r (University of Lubeck, Germany, see e.g., Zhang et al J. Med Chem 2020, Feb. 11, 2020), or darunavir, cobicistat or darunavir and cobicistat;
   (c) an angiotensin-converting enzyme 2(ACE 2 ) inhibitor, optionally to block the site of viral spike protein interaction for anti-SARS-CoV-2 infection control;
   (d) an anti-vascular endothelial growth factor (VEGF) (optionally VEGF A) drug or antibody, optionally bevacizumab;
   (e) a serine protease inhibitor, optionally camostat;
   (f) anti-PD-1 checkpoint inhibitor, optionally camrelizumab;
   (g) a compound or antibody capable of binding complement factor C5 and blocking membrane attack complex formation, optionally eculizumab;
   (h) a cathepsin inhibitor, optionally a cathepsin K, B or L inhibitor, optionally relacatib;
   (i) thalidomide, or thalidomide and glucocorticoid (optionally low-dose glucocorticoid), or and thalidomide and celecoxib,
   (j) an antibacterial antibiotic or a macrolide drug, wherein optionally the macrolide drug comprises azithromycin (e.g., ZITHROMAX™, or AZITHROCIN™), clarithromycin (e.g., BIAXIN™), erythromycin (e.g., ERYTHROCIN™), or fidaxomicin (e.g., DIFICID™ or DIFICLIR™), troleandomycin (e.g., TEKMISIN™), tylosin (e.g., TYLOCINE™ or TYLAN™), solithromycin (e.g., SOLITHERA™), oleandomycin (or SIGMAMYCINE™), midecamycin, roxithromycin, kitasamycin or turimycin, josamycin, carbomycin or magnamycin, and/or spiramycin; or
   (k) any combination of (a) to (j).

In alternative embodiments, provided are uses of a therapeutic combination of drugs or drug, a pharmaceutical dosage form, a drug delivery device, or a product of manufacture as provided herein for treating, preventing, ameliorating, slowing the progress of, decreasing the severity of or preventing a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales.

In alternative embodiments, provided are therapeutic combinations of drugs or a drug, a pharmaceutical dosage form, a drug delivery device, or a product of manufacture as provided herein for use in treating, preventing, ameliorating, slowing the progress of, decreasing the severity of or preventing a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales.

In alternative embodiments, provided are uses of a therapeutic combination of drugs or a drug, a pharmaceutical dosage form, a drug delivery device, or a product of manufacture as provided herein for the manufacture of a medicament,
wherein optionally the medicament is used for treating, preventing, ameliorating, slowing the progress of, decreasing the severity of or preventing a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales.

The details of one or more exemplary embodiments of the invention are set forth in the description below. Other features, objects, and advantages of the invention will be apparent from the description, figure and from the claims.
All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 illustrates an exemplary product of manufacture of the invention, an exemplary blister pack. As illustrated the “oseltamivir”, or yellow highlighted, section of the blister pack is intended to be taken by the a patient who may have been in contact with an individual infected with Coronavirus, such as an individual having a history of meeting or coming into contact with an infected person, or who may have been in contact with an individual already having a fever and/or a symptom of a respiratory illness such as cough or shortness of breath, but the patient as yet has not received the results of a blood test (or any diagnostic test) for Coronavirus. The patient given the exemplary blister pack, and while waiting for his or her test result immediately begins taking a high dose 5 times daily of oseltamivir (which should have few adverse effects on the patient), which should begin slowing the spread and/or replication of the virus in the patient (assuming the patient is in fact infected). As soon as the patient if confirmed by a blood test (or any diagnostic test) to be infected with Coronavirus, or after five days on treatment with oseltamivir, the patient immediately moves on to the next part (section) of the blister pack, which in alternative embodiment has three or more drugs, for example: lopinavir, ritonavir and oseltamivir; lopinavir combined (formulated) with ritonavir, or lopinavir and ritonavir separately formulated; lopinavir combined (formulated) with ritonavir, and oseltamivir; lopinavir and oseltamivir; ritonavir and oseltamivir; oseltamivir and remdesivir, and the like as provided herein. A doctor can decide the frequency of dosing based on the clinical presentation of the patient and test results. If the blood (or other) test is negative for coronavirus, the patient may return the blister pack or keep it (or keep taking the oseltamivir, until a second 2^{nd} test (which can be the same or a different test) is also negative.

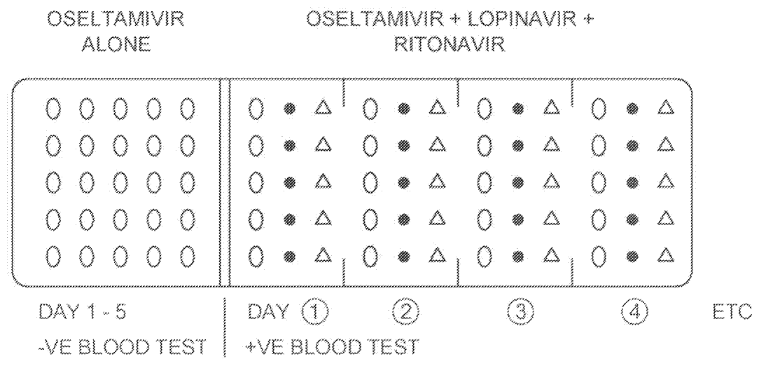

The drawings set forth herein are illustrative of exemplary embodiments provided herein and are not meant to limit the scope of the invention as encompassed by the claims.

Like reference symbols in the various drawings indicate like elements.

### DETAILED DESCRIPTION

In alternative embodiments, provided are pharmaceutical compositions comprising combinations of drugs, including products of manufacture and kits, and methods for using them, for treating, preventing (as a prophylaxis), ameliorating, slowing the progress of, decreasing the severity of or preventing a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales. In alternative embodiments, combinations, or cocktails, of drugs as provided herein are used to block intracellular metabolic pathways and intracellular viral replication, and prevent progression of the infection to clinical illness and death.

In alternative embodiments, provided are combinations of different medications which are used together can treat, ameliorate, slow the progress of, decrease the severity of or prevent the current (2019-nCoV) infections. In alternative embodiments, provided are novel methods of administration dosing to cover the period of exposure, diagnosis, and treatment.

In alternative embodiments, provided is combination of drugs comprising lopinavir combined (formulated) with ritonavir, or KALETRA™, ALTERA™, ALUVIA™, KALMELTREX, LOPIMUNE™ or LOPINAVIR™, and oseltamivir (or TAMIFLU™). In alternative embodiments, provided is combination of drugs comprising lopinavir, ritonavir and oseltamivir (or TAMIFLU™).

In alternative embodiments, provided are products of manufacture such as blister packs or equivalents (e.g., a clamshell, a tray, a shrink wrap and the like) comprising lopinavir combined (formulated) with ritonavir and oseltamivir, or lopinavir, ritonavir and oseltamivir. In alternative embodiments, the products of manufacture, e.g., blister pack, a clamshell, a tray, a shrink wrap and the like, arranges the drugs such that all drugs are taken together, or the oseltamivir is taken before the lopinavir combined (formulated) with ritonavir, or lopinavir and ritonavir.

In alternative embodiments, provided are methods for using combination of drugs and products of manufacture as provided herein comprising first administering to an individual (e.g., an individual suspected of being exposed to the coronavirus, e.g., an individual having a history of meeting or coming into contact with an infected person, or an individual already having a fever and/or a symptom of a respiratory illness such as cough or shortness of breath), oseltamivir (or TAMIFLU™) immediately and tests sent off for the coronavirus, with addition of (at least) lopinavir combined (formulated) with ritonavir, or lopinavir and ritonavir (separate formulations) to the administered drug regime when the test is positive for coronavirus (which can be within one, two, three or four or more days); and optionally also administering this regimen if the individual continues to have symptoms and is clinically judged to have coronavirus albeit (even if there is a) negative test.

Alternatively these drugs can be administered in the reverse order, i.e., first administer lopinavir combined (formulated) with ritonavir, or lopinavir and ritonavir (separate formulations), followed by (within one, two, three or four days, optionally if the individual continues to have symptoms) addition of administration of at least a third agent oseltamivir (or TAMIFLU™).

In alternative embodiments, dosing can be in the ratio of 25:100:75 of lopinavir: ritonavir: oseltamivir. For example, 25 mg (lopinavir), 100 mg (ritonavir), 75 mg (oseltamivir) respectively, or in multiples thereof, are administered one to ten times per day (e.g., bid, tid, or 5, 6, 7, 8, 9, or 10 or more times a day) depending on the stage of the condition (for example, exposed to infection, positivity in blood but asymptomatic, or symptomatic, mild or severe. In alternative embodiments, oseltamivir, lopinavir and ritonavir are administered in increased amounts, e.g., if the individuals condition does not improve, or does not improve quickly.

In alternative embodiments, products of manufacture as provided herein further comprise, or methods as provided herein further comprise use (administration of) efavirenz (e.g., SUSTIVA™), nevirapine (or the combination efavirenz with emtricitabine and tenofovir, or ATRIPLA™), amprenavir (e.g., AGENERASE™), nelfinavir (e.g., VIRACEPT™) and/or remdesivir (e.g., GS-5734™, Gilead Sciences). In alternative embodiments, one, several or all of these are concomitantly used in medications, e.g., one, several or all of these can also be used in (formulated with) a drug composition or formulation or product of manufacture as provided herein, or can be used or administered separately, alone or altogether, depending on the severity of the patient's illness.

In alternative embodiments, individuals or patients to whom a drug combination or composition or formulation as provided herein can be: (1) individuals or patients having been in contact with an infected person but are asymptomatic, or (2) individuals or patients that have been diagnosed by a blood test (i.e., are positive 20 for virus) but are asymptomatic, or (3) individuals or patients that are symptomatic, for example, that have fever, sore throat, cough, chest pain, dyspnea and/or diarrhea, or are severely ill with high fever, aches and pains, unable to breathe to walk and/or barely maintaining consciousness.

In alternative embodiments, a drug combination or composition or formulation as provided herein is administered prophylactically, e.g., to individuals who should or want to take the medication with them when travelling to prevent falling ill, e.g., to prevent acquiring the infection when away from their doctor, country, or language.

In alternative embodiments, a drug combination or composition or formulation as provided herein is packaged and/or administered as a product of manufacture such as a blister pack or equivalent. In alternative embodiments, the blister pack or equivalent is designed to cover various stages of the infection, or to be for prophylactic purposes.

Alternatively the compounds may be solubilized, and then filtered with 22 micron filters or equivalents, suspended in a sterile fashion in saline or water or equivalents and administered intravenously, e.g., in emergencies.

In alternative embodiments, methods comprise formulating and/or administering chloroquine (e.g., ARALEN™), chloroquine phosphate, chloroquine diphosphate, hydroxychloroquine (e.g., PLAQUENIL™) intravenously at about 300 mg, or between 100 mg and 500 mg, in single dosages, or the equivalent thereof as an infusion.

In alternative embodiments, methods comprise formulating and/or administering chloroquine (e.g., ARALEN™), chloroquine phosphate, chloroquine diphosphate, hydroxychloroquine (e.g., PLAQUENIL™) at about 2.5 mg/kg intramuscularly (IM) at e.g., 0, 1, 12, 23, 24 and 25 hours, or e.g., at one or multiple dosages for between about one to two days or one day to two weeks.

In alternative embodiments, methods comprise formulating and/or administering chloroquine (e.g., ARALEN™), chloroquine phosphate, chloroquine diphosphate, hydroxychloroquine (e.g., PLAQUENIL™) at about 5 mg/kg subcutaneously (SC), e.g., at 0, 12 and 24 hours, or e.g., at one or multiple dosages for between about one to two days or one day to two weeks.

In alternative embodiments, oral administration of chloroquine (e.g., ARALEN™), chloroquine phosphate, chloroquine diphosphate, hydroxychloroquine (e.g., PLAQUENIL™) follows or complements (e.g., is delivered together with) the IM or SC administration, or with the aerosol or powder administration of chloroquine (e.g., ARALEN™), chloroquine phosphate, chloroquine diphosphate, hydroxychloroquine (e.g., PLAQUENIL™), as described below. In alternative embodiments, oral administration is dosaged at about 5 mg/kg, e.g., for between about 12 and 72 hours (h), or for between about 36 and 48 h.

In alternative embodiments, methods comprise first administering (alone) for about 7 days (or 2, 3, 4, 5, 6 or 7 or more days) oseltamivir (or TAMIFLU™) at 3 times per day (tid) (or alternatively twice a day (bid) or four times a day or more), followed by a blood sample to be taken for virus testing; this initial administration dampens or slows a possible virus infection developing in the individual, so possibly ending up with a milder disease course, e.g., where the side effects would be milder.

If and when virus blood positivity is confirmed, or viral infection is otherwise confirmed, the osteltamivir is supplemented by the lopinavir and ritonavir (which can be administered together or separately) three times daily (tid) (or alternatively twice a day (bid) or four times a day or more), while continuing the osteltamivir, for example, all three medications three (or two or four or more) times daily or 9 (or more) medications daily.

In alternative embodiments, the duration of the combined drug therapy as provided herein is 2, 3, 4, 5, 6, 7, 8, 9 or 10 or more days, which can be prolonged in those whose blood coronavirus remains positive longer with daily blood tests (or equivalent tests to confirm continued active infection), until the infection is shown to be gone or substantially diminished, particularly when the patient has symptomatically otherwise substantially recovered.

In alternative embodiments, other anti-coronavirus medications e.g., listed above (e.g., efavirenz (e.g., SUSTIVA™), nevirapine (or the combination efavirenz with emtricitabine and tenofovir, or ATRIPLA™), amprenavir (e.g., AGENERASE™), nelfinavir (e.g., VIRACEPT™) and/or remdesivir (e.g., GS-5734™, Gilead Sciences)) are added or mixed into the ‘cocktail’, e.g., are mixed into osteltamivir, lopinavir and/or ritonavir formulations or one, several or all are administered separately.

In alternative embodiments, the contents of a blister pack or equivalent as provided herein have arranged thereof a combination of drugs (e.g., as pill, capsules, tablets) to facilitate the patient's self-administration of a drug regimen as provided herein.

In alternative embodiments, an individual (e.g., a patient) is given one, several or all of these medications (as provided in drug combinations or formulations as provided herein or used in methods as provided herein) in the form of a tablet, a capsule, a liquid, a spray, a powder, via an enema, as a suppository, administered subcutaneously or intravenously where available. When the patient is on a life support system the drug combination can be given parenterally.

### Products of Manufacture and Kits

Provided are products of manufacture and kits for practicing methods as provided herein; and optionally, products of manufacture and kits can further comprise instructions for practicing methods as provided herein.

Provided are compositions, including preparations, formulations and/or kits, comprising combinations of ingredients, e.g., therapeutic combinations as described herein. In alternative embodiments, therapeutic combination can be mixed and administered together, or alternatively, they can be an individual member of a packaged combination of ingredients, e.g., a liquid component and a solid product component manufactured in a separate compartment, package, kit or container; e.g., where all or a subset of the combinations of ingredients are manufactured in a separate compartment, package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, provided are for blister packs, clamshells or trays comprising a drug combination or formulation as provided herein, or a drug combination, pharmaceutical preparations or pharmaceutical compositions used to practice methods as provided herein. Blister packs, clamshells or trays can be designed to be non-reclosable, so consumers can tell if a package has already opened. They are used to package for sale goods where product tampering is a consideration, such as the pharmaceuticals as provided herein. In one aspect, a blister pack comprises a moulded PVC base, with raised areas (the “blisters”) to contain the tablets, pills, etc. comprising the combinations of drugs drug combination, or formulations, pharmaceutical preparations or pharmaceutical compositions used in methods as provided herein, covered by a foil laminate. Tablets, pills, etc. can be removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one embodiment, provided is a method of packaging wherein the compositions comprising combinations of ingredients are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two or three or more components: a thermoformed “blister” which houses multi-ingredient combination as provided herein, and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (e.g., using an AERGO 8 DUO™, SCA Consumer Packaging, Inc., DeKalb IL) using regular heat seal tooling. This alternative aspect, using heat seal tooling, can seal common types of thermoformed packaging.

In alternative embodiments, therapeutic combinations and formulations drug combination, or pharmaceutical preparations or pharmaceutical compositions used in methods drug combination, are formulated, e.g., as a powder, e.g., as lyophilised material, e.g., a lyophilized encapsulated product, e.g., for practicing methods as provided herein, can be packaged alone or in combinations, e.g., as “blister packages” or as a plurality of packettes, including as lidded blister packages, lidded blister or blister card or packets or packettes, or a shrink wrap.

In alternative embodiments, laminated aluminium foil blister packs are used, e.g., for the preparation of therapeutic combinations or formulations as provided herein, or for pharmaceutical preparations or pharmaceutical compositions used in methods as provided herein. Products or kits comprise an aqueous solution(s) which are dispensed (e.g., by measured dose) into containers. Trays can be freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, e.g., using hard temper aluminium (e.g., alufoil) lidding material. In one aspect, hermetically-sealed high barrier aluminium (e.g., alufoil) laminates are used. In one aspect, products of manufacture include kits or blister packs, use foil laminations and strip packs, stick packs, sachets and pouches, peelable and non-peelable laminations combining foil, paper, or film for high barrier packaging.

In alternative embodiments, multi-component products of manufacture, including kits or blister packs as provided herein, include memory aids to help remind patients when and how to take the therapeutic combination. This safeguards the therapeutic combination's efficacy by protecting each tablet, geltab or pill until it's taken; gives the product or kit portability, makes it easy to take a dose anytime or anywhere.

### Inhalers and Nasal Sprays

In alternative embodiments, provided are drug delivery devices comprising an inhalation device or inhaler or a nasal spray device for the delivery of a therapeutic combination of drugs, a pharmaceutical dosage form or a formulation as provided herein. In alternative embodiments, provided are methods for administering a therapeutic combination of drugs, a pharmaceutical dosage form or a formulation as provided herein using an inhalation device or inhaler or a nasal spray device, e.g., for the delivery of chloroquine (e.g., ARALEN™), chloroquine phosphate, chloroquine diphosphate, hydroxychloroquine (e.g., PLAQUENIL™), lopinavir, ritonavir and/or oseltamivir, and/or an anti-viral drug or medication or an anti-microbial drug as provided herein, or an anti-viral or an anti-microbial drug as used to practice methods as provided herein.

In alternative embodiments, the inhaler or the nasal spray device is a hand-held or otherwise portable inhaler or a nasal spray device, and optionally the inhaler or a nasal spray device is a metered or dose-counting inhaler or a nasal spray device. In alternative embodiments, the inhaler or the nasal spray device is a device as described in e.g., U.S. Pat. Nos. 10,583,261, or 10,561,809 (describing a breath actuated dry powder inhaler with a single air circulation chamber for de-agglomeration of entrained powdered medicament), or U.S. Pat. No. 10,561,807 (describing inhaler devices configured for consuming a defined capacity and generate an aerosol or aerosol imparted with flavor, a sensor configured to detect a predefined variable, an interface configured to make a notification to an inhaler of the aerosol, and a controller), or U.S. Pat. No. 10,463,815 (describing a dry powder inhaler may include a powder storage region, an inlet channel, a dispersion chamber, and an outlet channel); or U.S. patent application publication no. 20200069897 (describing inhalers having a breath actuated trigger mechanism reactive to an inhalation flow to trigger the release of a substance to be inhaled); or 20200061314 (describing a smart inhaler device having a flow pathway comprising a cartridge receptacle that is able to house a cartridge, flow meter, pump, and vaporizer; a wireless communication module; and at least one sensor that captures identifying information related to the cartridge); or 2020004691 (describing dry powder inhalers having replaceable cartridges containing a dry powder for local or systemic delivery through the pulmonary tract and lungs); or 20200046916 (describing an inhaler having a refill assembly comprising: a patient port; a canister actuable by the reusable assembly to deliver a dose of medicament to the patient port, a sleeve which is selectively actuable by a user independently of the reusable assembly so as to act on the canister to deliver a dose of medicament); or 20200046029 (describing an apparatus for generating an aerosol and/or a vapour in an inhaler device includes a reservoir for storing a supply of a liquid; a heating system fluidly connected with the reservoir for receiving the liquid and configured to heat the liquid to generate the aerosol and/or vapor therefrom; a pumping system configured to pump the liquid from the reservoir to the heating system; and a valve arrangement for regulating flow from the pumping system to the heating system); or 20200016345 (describing a dry powder inhaler having a first chamber having an orifice for holding a dry powder and a gas, and a second chamber directly connected to the first chamber by at least one passageway for receiving an aerosolized form of the dry powder from in the first chamber and delivering the aerosolized dry powder to a user). An inhaler as provided herein, or as used in methods as provided herein, can comprise use of a dose counter, e.g., as described in U.S. Pat. No. 10,561,808.

In alternative embodiments, the inhaler or the nasal spray device is a handheld or otherwise portable inhaler or a nasal spray device is used or intended for use on public transport such as buses, trams, trains, aircraft and/or boats, or in places of commerce such as stores, bars, sporting events, movies theaters, theater, musical events, or any gathering of people.

In alternative embodiments, the chloroquine (e.g., ARALEN™), chloroquine phosphate, chloroquine diphosphate, hydroxychloroquine (e.g., PLAQUENIL™), lopinavir, ritonavir and/or oseltamivir, and/or the anti-viral drug or drug combination or medication as provided herein, or anti-microbial drug as provided herein, is formulated as a powder (e.g., a dry powder), a microparticle or a nanoparticle, or an aerosol. In alternative embodiments, the powder can be an agglomeration of powder particles or an agglomerate having irregular geometries such as width, diameter, and length. In alternative embodiments, the dry powder can be formulated as a granule of a physiologically acceptable excipient to be used as a carrier for a dry powder formulation for inhalation as described e.g., in U.S. Pat. No. 10,583,085.

In alternative embodiments, the chloroquine (e.g., ARALEN™), chloroquine phosphate, chloroquine diphosphate, hydroxychloroquine (e.g., PLAQUENIL™) is formulated at between about 50% to 100% concentration as a liquid or aqueous formulation. In alternative embodiments, the chloroquine (e.g., ARALEN™), chloroquine phosphate, chloroquine diphosphate, hydroxychloroquine (e.g., PLAQUENIL™) is formulated and delivered at a dosage regimen of about 0.2 mg/kg to about 150 mg/kg per dose.

In alternative embodiments, methods of delivery of the chloroquine (e.g., ARALEN™), chloroquine phosphate, chloroquine diphosphate, hydroxychloroquine (e.g., PLAQUENIL™), lopinavir, ritonavir and/or oseltamivir, and/or the anti-viral drug or medication, or anti-microbial drug, comprise treatment regimens where the drug, medication or combination of drugs are administered every hour, every other hour, once, twice, three, four, five, six, seven, eight, nine, ten, eleven or twelve times a day. In alternative embodiments, the length of time of treatment, or the exact dosaging or dosage regimen, is determined by the clinician, or the administration is to begin immediately after possible exposure to an individual having (or exposed to another individual having) a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales.

In alternative embodiments, the chloroquine (e.g., ARALEN™), chloroquine phosphate, chloroquine diphosphate, hydroxychloroquine (e.g., PLAQUENIL™) is formulated at between about 50% to 100% concentration as a liquid or aqueous formulation, which can be used either as an aerosol and/or given orally. In alternative embodiments, the chloroquine (e.g., ARALEN™), chloroquine phosphate, chloroquine diphosphate, hydroxychloroquine (e.g., PLAQUENIL™) is formulated and delivered (e.g., by inhalation and/or orally) at a dosage regimen of about 0.2 mg/kg to about 150 mg/kg per dose.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of”or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of'', and ”consisting of'′ may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

### Example 1: Exemplary Treatment Regimens

This example demonstrates that methods and products of manufacture as provided are effective for treating, preventing, ameliorating, slowing the progress of, decreasing the severity of or preventing a coronavirus infection, or a COVID-19 (or so-called Wuhan coronavirus) infection, or an infection caused by a virus in the subfamily Orthocoronavirinae, or a virus in the family Coronaviridae, or a virus in the order Nidovirales.

A 42 year (y) old female patient is first treated with osteltamivir (or TAMIFLU™) from the time she was in contact with a patient from China later found to be positive for a coronavirus, in particular, the COVID-19 virus (or so-called Wuhan coronavirus). She is treated for 4 days but then develops fever, cough, aches and some dyspnea, with blood result coming back positive for COVID-19 virus.

Because the patient remains positive for 2019-nCoV virus, treatment with lopinavir and ritonavir, or KALETRA™, ALTERA™, ALUVIA™, KALMELTREX, LOPIMUNE™ or LOPINAVIR™, is now added to the osteltamivir treatment, and within 48 hours her blood test becomes negative for the coronavirus.

This patient was quite ill reaching the dyspnoea phase and difficulty walking, and but for this new drug combination treatment would have been expected to die. The combination of drugs as provided herein can rapidly clear the coronavirus from the patient's blood, thereby terminating or significantly ameliorating an otherwise life-threatening illness.

### Example 2: Exemplary Treatment Regimen

A 47-year-old male returning from a trip develops draggles and muscle pains and a temperature of 37.5 C. He is tested for coronavirus and is found positive for COVID-19 on a swab test. He is commenced on a combination of twice-daily chloroquine to 250 mg, lopinavir 200 mg bid, retinovir 50 mg bid. together with 75 mg bid oseltamivir (e.g., TAMIFLU™).

His condition is further resolved within three days and he is negative on the swab test on day six. Muscle pains also disappear and he feels well with and is able to be released to go home from hospital on day seven.

### Example 3: Exemplary Treatment Regimen

A group of elderly travelers board a ship on a cruise, whereupon numerous individuals on the ship contract coronavirus COVID-19 on testing. This group is administered as a prophylactic therapy/treatment inhalant agents comprising two inhaled doses, or twice daily, of 125 mg of chloroquine. None contract COVID-19 coronavirus and test negative for the virus upon arrival home after the cruise.

### Example 4: Exemplary Treatment Regimen

A 65-year-old female patient develops a very spiritual section with shortness of breath, and is admitted to a hospital, and is tested positive for COVID-19 coronavirus. She is treated with intravenous (IV) remdesivir 10 mg per kilogram, chloroquine 250 mg twice daily, inhaled interferon, and KALETRA™ (a lopinavir/ritonavir combination) 50/200 mg.

Over the next week the patient's shortness of breath first worsens, but then improves, having fewer muscle aches and shortness of breath. By day 8 her viral detection is negative for COVID-19. The patient is discharged on day 12 fully cured of the coronavirus using this exemplary drug combination.

### Example 5: Exemplary Treatment Regimen

Two Italian patients (one female, one male 69 years old with previous pulmonary disease) traveling in India tested positive for coronavirus COVID-19; they were administered lopinavir 200 mg twice daily, ritonavir 50 mg twice daily, chloroquine 250 mg twice daily, 75 mg oseltamivir (TAMIFLU™) twice daily, and the female patient tested negative after 7 days of this combined drug therapy, and the male patient showed significant improvement, with COVID-19 viral load diminished. No significant side effects from the administered drug combination were seen.

### Example 6: Exemplary Treatment Regimen

A 32-year-old male patient acquires nose swab positive coronavirus COVID-19 infection, possibly at a party. Symptoms include loss of the sense of taste, muscle aches, fever of 38.9C, sore throat cough and difficulty with breathing. He is seen by a COVID-19 specialist, who commences patient on a combination of opaganib or YELIVA™ tid at 200 mg or 300 mg per dose; hydroxychloroquine 200 mg twice daily; lopinavir 200 mg three times per day; ritonavir 50 mg three times per day, and oseltamivir 75 mg tid, where one, several or all of these drugs are administered orally, IM, IV and/or by inhalation individually or in combination (e.g., as a single formulation where applicable). The patient progressively loses his fever, regains the sense of taste, and after further five days the cough and sore throat improve. Shortness of breath progressively improves but this requires more than 20 days (d) of continued treatment, yet the swabs are negative for COVID-19 on consecutive days by day 8.

### Example 7: Exemplary Treatment Regimen

A 72-year-old gentleman with a chronic cough suddenly develops fever, worsening of cough and shortness of breath walking up hills. He goes to the emergency room but the antibiotic that is given does not stop the progression of the illness. He sees a specialist who deals with respiratory disease and is found to be positive for COVID-19. He is commenced on opaganib or YELIVA™ tid at 200 mg or 300 mg per dose; and hydroxychloroquine 200 mg tid. He continues on this therapy and notices improvement within two days; a swab is negative on day seven and he is completely asymptomatic by day 20.

### Example 8: Exemplary Treatment Regimen

A 27-year-old female patient has a nose swab positive coronavirus COVID-19 infection. Symptoms include myalgia, sore throat cough and difficulty with breathing. She also complains of marked fatigue. The doctor commences her on a combination of hydroxychloroquine 200 mg twice daily (bid), lopinavir 200 mg three times per day (tid), ritonavir 50 mg tid, and oseltamivir 75 mg tid. The patient loses her fever after four days although the cough and sore throat continues for another two days. Shortness of breath progressively improves and after 12 days of treatment the swabs become negative on consecutive days.

### Example 9: Exemplary Treatment Regimen

A 73-year-old male develops loss of sense of smell and taste, followed by muscle aches and pains fever of 38.5° C. cough and a sore throat. He then notices he was short of breath and saw his doctor. The doctor commences him on hydroxychloroquine 200 mg twice daily increasing after three days to 3 times daily, together with azithromycin 50 mg three times a day, and within three days the patient's fever improves, then disappears. The patient continues on this composition of the two drugs for a total of 12 days and subsequent swabs are negative for coronavirus COVID-19.

### Example 10: Exemplary Treatment Regimen

A 49-year-old patient with nasopharyngeal swab proving an infection with coronavirus COVID-19, is treated to reduce symptoms using a combination of opaganib or YELIVA™ tid at 200 mg or 300 mg per dose, hydroxychloroquine 200 mg tid, azithromycin 50 mg bid. On day 4 of the treatment nasal swabs show an absence of the coronavirus, and this is also shown daily until day 14 showing a cure is achieved. The patient's symptoms abate fairly rapidly and he eventually becomes completely normal and asymptomatic.

### Example 11: Exemplary Prophylactic Treatment Regimens

Individuals are given an initial loading dosage of chloroquine, chloroquine phosphate, chloroquine diphosphate and/or hydroxychloroquine, which is administered or started at a high dose (e.g., the so-called “loading dose”) for example, an oral (e.g., a single dose such as a single tablet, capsule or pill), IV or IM dosage of between about 250 mg, 300 mg, 350 mg, 300 mg or 0.5 gram (gm) (500 mg) and 1.5 gm, or between about 400 mg and 1 gm, optionally with follow up administrations every 4 to 10 days, or every 1, 2, 3, 4, 5, 6, 7, 8, 9 or 10 days, at a lower dosage of between about 50 gm to 200 mg, or about 100 mg, total daily dosage, optionally continuing for between about one week to one month,
and the chloroquine, chloroquine phosphate, chloroquine diphosphate and/or hydroxychloroquine is administered together with:
   a macrolide drug, optionally azithromycin, and optionally the macrolide drug is started with a high dose (e.g., a so-called “loading dose”), optionally an oral (e.g., a single dose such as a single tablet, capsule or pill), IV or IM dosage of between about 400 mg to 0.5 gram (gm) (500 mg) and 1 gm, or at about 500 mg, optionally with follow up administrations every 4 to 10 days, or every 1, 2, 3, 4, 5, 6, 7, 8, 9 or 10 days, at a lower dosage of between about 100 gm to 300 mg, or about 250 mg, total daily dosage, optionally continuing for between about one week to one month, and/or
   opaganib or YELIVA™, wherein optionally the opaganib is administered at a dosage of QD (once a day), bid (twice a day) or tid (three times a day) at a dosage of between about 100 to 600 mg per day or per dosage, or at about 100, 200, 300, 400, 500 or 600 mg per day or per dosage, and/or
lopinavir combined (formulated) with ritonavir, or KALETRA™, ALTERA™, ALUVIA™, KALMELTREX, LOPIMUNE™ or LOPINAVIR™.

A number of embodiments of the invention have been described. Nevertheless, it can be understood that various modifications may be made without departing from the spirit and scope of the invention. Accordingly, other embodiments are within the scope of the following claims.

What is claimed is:

1. A therapeutic combination of drugs or a drug, a pharmaceutical dosage form, a drug delivery device, or a product of manufacture, comprising:
(a) opaganib, or YELIVA™, or opaganib, or YELIVA™ and oral and/or inhaled chloroquine (or ARALEN™), chloroquine phosphate, chloroquine diphosphate and/or hydroxychloroquine (e.g., PLAQUENIL™);
(b) lopinavir, ritonavir and oseltamivir (e.g., TAMIFLU™);
(c) lopinavir combined (formulated) with ritonavir, or KALETRA™, ALTERA™, ALUVIA™, KALMELTREX, LOPIMUNE™ or LOPINAVIR™, or lopinavir and ritonavir separately formulated;
(d) lopinavir combined (formulated) with ritonavir (or KALETRA™, ALTERA™, ALUVIA™, KALMELTREX, LOPIMUNE™ or LOPINAVIR™), or lopinavir and ritonavir, and oseltamivir (e.g., TAMIFLU™), optionally also with inhaled formulations or versions of chloroquine (or ARALEN™), chloroquine phosphate, chloroquine diphosphate and/or hydroxychloroquine (e.g., PLAQUENIL™) and/or oral chloroquine (or ARALEN™), chloroquine phosphate, chloroquine diphosphate and/or hydroxychloroquine (e.g., PLAQUENIL™) simultaneously;
(e) lopinavir, ritonavir, chloroquine and oseltamivir (or TAMIFLU™); wherein optionally the chloroquine comprises inhaled chloroquine (or ARALEN™), chloroquine phosphate, chloroquine diphosphate and/or hydroxychloroquine (e.g., PLAQUENIL™) and/or oral chloroquine (or ARALEN™), chloroquine phosphate, chloroquine diphosphate and/or hydroxychloroquine (e.g., PLAQUENIL™) simultaneously;
(f) lopinavir and oseltamivir (e.g., TAMIFLU™);
(g) ritonavir and oseltamivir (e.g., TAMIFLU™);
(h) remdesivir (e.g., GS-5734™, Gilead Sciences) alone, or oseltamivir (e.g., TAMIFLU™) and remdesivir (e.g., GS-5734™, Gilead Sciences), and optionally the remdesivir is an oral formulation and/or an inhaled remdesivir formulation;
(i) oseltamivir (e.g., TAMIFLU™) and efavirenz (e.g., SUSTIVA™);
(j) oseltamivir (e.g., TAMIFLU™) and nevirapine (or the combination efavirenz with emtricitabine and tenofovir, or ATRIPLA™);
(k) oseltamivir (or TAMIFLU™) and amprenavir (e.g., AGENERASE™);
(l) oseltamivir (e.g., TAMIFLU™) and nelfinavir (e.g., VIRACEPT™); or
(m) a thiazolide class drug, optionally nitazoxanide (or ALINIA™, NIZONIDE™) or tizoxanide (or 2-Hydroxy-N-(5-nitro-2-thiazolyl)benzamide), with or in combination with any of (a) to (i), or the thiazolide class drug (e.g., nitazoxanide or tizoxanide) and oseltamivir (or TAMIFLU™);
(n) plitidepsin (also known as dehydrodidemnin B), or APLIDIN™ (PharmaMar, S.A.);
(o) an inhibitor or S-phase kinase-associated protein 2(SKP 2 ), or dioscin, or niclosamide, or NICLOCIDE™, FENASAL™, or PHENASAL™;
(p) ribavirin, interferon beta 1b, or a combination of ribavirin and interferon beta, or a combination of lopinavir and ritonavir and interferon-beta-lb;
(q) abacavir, actemra, acyclovir e.g., (ACICLOVIR™), adefovir, amantadine, ampligen, amprenavir (e.g., AGENERASE™), aprepitant, arbidol, atazanavir, atripla, balavir, baloxavir marboxil (XOFLUZA™), bepotastine, bevirimat, bictegravir, biktarvy, brilacidin, cidofovir, caspofungin, lamivudine and zidovudine (e.g., COMBVIR™), cobicstat, colisitin, cocaine, darunavir, delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II and/or interferon type III), lamivudine, lopinavir, loviride, ledipasvir, leronlimab, maraviroc, methisazone, moroxydine, nelfinavir, nevirapine, nexavir, nitazoxanide, norvir, a nucleoside analogue (optionally brincidofovir, didanosine, favipiravir (also known as T-705, avigan, or favilavir, Toyama Chemical, Fujifilm, Japan), vidarabine, galidesivir (e.g., BCX4430, IMMUCILLIN-A™), remdesivir (e.g., GS-5734™, Gilead Sciences), cytarabine, gemcitabine, emtricitabine, lamivudine, zalcitabine, abacavir, acyclovir, entecavir, stavudine, telbivudine, zidovudine, idoxuridine and/or trifluridine or any combination thereof), oseltamivir (or TAMIFLU™), peginterferon alfa-2a, penciclovir, peramivir (e.g., RAPIVAB™), perfenazine, pleconaril, plurifloxacin, podophyllotoxin, pyramidine, raltegravir, rifampicin, ribavirin, rilpivirine, rimantadine, ritonavir, saquinavir, sofosbuvir, stavudine, telaprevir, tegobuv, tenofovir alafenamide, tenofovir disoproxil, tenofovir, tipranavir, trifluridine, trizivir, tromantadine, truvada, valaciclovir (e.g., VALTREX™), valganciclovir, valrubicin, vapreotide, vicriviroc, vidarabine, viramidine, velpatasvir, vivecon, zalcitabine, zanamivir (e.g., RELENZA™), zidovudine or any combination thereof;
(r) an mucolytic therapy or drug, optionally acetylcysteine, ambroxol, bromhexine, carbocisteine, erdosteine, mecysteine or domase alfa, or an expectorant, optionally guaifenesin;
(s) a viral, or a coronavirus or a COVID-19, protease inhibitor, optionally ASC09 (CAS registry no. 1000287-05-7) (Janssen Research and Development, LLC), ritonavir or ASC09 and ritonavir, or a JAK1/2 inhibitor (optionally baricitinib), optionally compound 11r (University of Lubeck, Germany, see e.g., Zhang et al J. Med Chem 2020, Feb. 11, 2020), or darunavir, cobicistat or darunavir and cobicistat;
(t) an angiotensin-converting enzyme 2(ACE 2 ) inhibitor, optionally to block the site of viral spike protein interaction for anti-SARS-CoV-2 infection control;
(u) an anti-vascular endothelial growth factor (VEGF) (optionally VEGF-A) drug or antibody, optionally bevacizumab;
(v) a serine protease inhibitor, optionally camostat;
(w) anti-PD-I checkpoint inhibitor, optionally camrelizumab;
(x) a compound or antibody capable of binding complement factor CS and blocking membrane attack complex formation, optionally eculizumab;
(y) a cathepsin inhibitor, optionally a cathepsin K, B or L inhibitor, optionally relacatib;
(z) thalidomide, or thalidomide and glucocorticoid (optionally low-dose glucocorticoid), or and thalidomide and celecoxib;
(aa) an antibacterial antibiotic or a macrolide drug, wherein optionally the macrolide drug comprises azithromycin (e.g., ZITHROMAX™, or AZITHROCIN™), clarithromycin (e.g., BIAXIN™), erythromycin (e.g., ERYTHROCIN™), or fidaxomicin (e.g., DIFICID™ or DIFICLIR™), troleandomycin (e.g., TEKMISIN™), tylosin (e.g., TYLOCINE™ or TYLAN™), solithromycin (e.g., SOLITHERA™), oleandomycin (or SIGMAMYCINE™), midecamycin, roxithromycin, kitasamycin or turimycin, josamycin, carbomycin or magnamycin, and/or spiramycin;
(bb) chloroquine (or ARALEN™), chloroquine phosphate, chloroquine diphosphate and/or hydroxychloroquine (e.g., PLAQUENIL™) with (or formulated with) or in combination with any of (a) to (aa), or chloroquine, chloroquine phosphate, chloroquine diphosphate and/or hydroxychloroquine (e.g., PLAQUENIL™) and oseltamivir (or TAMIFLU™);
(cc) chloroquine (e.g., ARALEN™), chloroquine phosphate, chloroquine diphosphate and/or hydroxychloroquine (e.g., PLAQUENIL™);
(dd) any one or several or all of (a) to (cc) with (or formulated with or formulated as an) inhaled formulation and/or formulated with or formulated as an oral, intramuscular (IM) or intravenous (IV) formulation, wherein optionally both the inhaled and the oral, IV and/or IM formulations are administered simultaneously or sequentially, and optionally the inhaled formulation comprises chloroquine (or ARALEN™), chloroquine phosphate, chloroquine diphosphate and/or hydroxychloroquine (e.g., PLAQUENIL™) and/or oral chloroquine (or ARALEN™), chloroquine phosphate, chloroquine diphosphate and/or hydroxychloroquine (e.g., PLAQUENIL™) administered simultaneously or overlapping; or
(ee) any combination of (a) to (dd).

2. The therapeutic combination of drugs or drug, pharmaceutical dosage form, drug delivery device, or product of manufacture of claim 1, further comprising (or are formulated with) an (or an additional) anti-viral drug or medication, or antimicrobial drug, or palliative agent or drug,
wherein optionally the anti-viral drug or medication, or anti-microbial drug, is or comprises efavirenz (e.g., SUSTIVA™), tenofovir, emtricitabine and tenofovir, nevirapine (or the combination efavirenz with emtricitabine and tenofovir, or ATRIPLA™), amprenavir (e.g., AGENERASE™), nelfinavir (e.g., VIRACEPT™) and/or remdesivir (e.g., GS-5734™, Gilead Sciences), a viral RNA-dependent RNA polymerase inhibitor, optionally galidesivir,
and optionally the anti-viral drug or medication is or comprises an anti-retroviral drug or drug combination, and optionally the anti-retroviral drug or drug combination comprises: darunavir and cobicistat (e.g., REZOLSTA™ or PREZCOBIX™); atazanavir and cobicistat (or EVOTAZ™); abacavir, lamivudine and dolutegravir (TRIUMEQ™); tenofovir (or disoproxil or emtricitabine) and elvitegravir and cobicistat (e.g., STRIBILD™); tenofovir (or disoproxil or emtricitabine) and elvitegravir and cobicistat (COMPLERA™ or EVIPLERA™); efavirenz, emtricitabine and tenofovir (ATRIPLA); lamivudine, nevirapine and stavudine (e.g., TRIOMUNE™); atazanavir and cobicistat (e.g., EVOTAZ™); lamivudine and raltegravir (e.g., DUTREBIS™); lamivudine and dolutegravir (or DOVATO™); doravirine, lamivudine and tenofovir (e.g., DELSTRIGO™); or lamivudine, zidovudine and nevirapine (e.g., CUOVIR-N™);
and optionally the additional anti-viral drug or medication, or anti-microbial drug, is formulated with the chloroquine (e.g., ARALEN™), chloroquine phosphate, chloroquine diphosphate, hydroxychloroquine (e.g., PLAQUENIL™), lopinavir, ritonavir and/or oseltamivir or is formulated separately from the chloroquine (e.g., ARALEN™), chloroquine phosphate, chloroquine diphosphate, hydroxychloroquine (e.g., PLAQUENIL™), lopinavir, ritonavir and/or oseltamivir,
and optionally the anti-viral drug or medication, or anti-microbial drug, or palliative agent comprises zinc or vitamin C, which optionally is given enterally or parenterally.

3. The therapeutic combination of drugs or drug, pharmaceutical dosage form, drug delivery device, or product of manufacture of claim 1, wherein:
(a) the chloroquine (e.g., ARALEN™), chloroquine phosphate, chloroquine diphosphate, hydroxychloroquine (e.g., PLAQUENIL™), lopinavir, ritonavir and/or oseltamivir are formulated separately or together, or the lopinavir and ritonavir are formulated together and the oseltamivir is formulated separately;
(b) the therapeutic combination of drugs or drug, pharmaceutical dosage form, or the chloroquine (e.g., ARALEN™), chloroquine phosphate, chloroquine diphosphate, hydroxychloroquine (e.g., PLAQUENIL™), lopinavir, ritonavir and/or oseltamivir and/or the anti-viral drug or medication, or anti-microbial drug are formulated or contained in a liquid formulation (optionally sterile saline or water), a spray, a powder, an aerosol, or any formulation for inhalation, a pill, a capsule, a tablet, or a geltab, or equivalents,
and optionally the therapeutic combination of drugs or drug, pharmaceutical dosage form, or the chloroquine (e.g., ARALEN™), chloroquine phosphate, chloroquine diphosphate, hydroxychloroquine (e.g., PLAQUENIL™), lopinavir, ritonavir and/or oseltamivir and/or the anti-viral drug or medication, or anti-microbial drug, are coated on the surface of or contained in: a bead, a powder, a particle, or a multilayered bead or particle, and optionally the bead, powder, particle or the multilayered bead or particle is contained in a pill, a capsule, a tablet, or a geltab, or equivalents, for oral delivery,
wherein optionally the pill, capsule, tablet, geltab or equivalent for oral delivery is a hard gelatin capsule or equivalent, or comprises a hard gelatin or equivalent.

4. The therapeutic combination of drugs or drug, pharmaceutical dosage form, drug delivery device, or product of manufacture of claim 1, wherein the lopinavir, ritonavir and oseltamivir are formulated or packaged for administration as or dosing in the ratio of 25:100:75 of lopinavir: ritonavir: oseltamivir.

5. A drug delivery device or package, a kit, a blister package, a clamshell or a tray, comprising a therapeutic combination of drugs or drug, a pharmaceutical dosage form or a formulation of claim 1,
wherein the drug delivery device comprises an inhalation device or inhaler or a nasal spray device, and optionally the inhaler or a nasal spray device is a hand-held inhaler or a nasal spray device, and optionally the inhaler or a nasal spray device is a metered or dose-counting inhaler or a nasal spray device,
wherein the drug delivery device or package, blister pack, clamshell or tray comprises a plurality of compartments spatially arranged on the drug delivery device or package, blister pack, clamshell or tray to follow a dosage administration regimen,
wherein the spatially arranged plurality of compartments are in at least two rows, each row marked for the time for which a drug of the drug combination, or the tablets, pills, capsules, geltabs or equivalents, are to be taken by a user (optionally a patient), optionally one row marked for morning, breakfast or AM administration, and one row marked for evening, dinnertime or PM administration, and optionally the row or rows marked for morning, breakfast or AM administration is or are positioned above the row or rows marked for evening, dinnertime or PM administration,
and optionally the spatially arranged plurality of compartments are in four rows, two rows marked for morning, breakfast or AM administration, and two rows marked for evening, dinnertime or PM administration,
and optionally the spatially arranged plurality of compartments are arranged to facilitate and/or direct the patient to take the drugs in that row two times a day (bid), three times a day (tid), four times a day, or up to ten times a day (wherein optionally the higher amounts are for the very sick), optionally also comprising indication of actual times for patient to ingest the drugs in each row, and if appropriate, directions to also ingest fluids or other drugs and/or food (e.g., small amounts of food),
and optionally each row comprises seven compartments for one dosage administration for each day of the week, or eight compartments for one dosage administration for each day of the week and one spare, and optionally each vertically arranged set of compartments, or columns, are marked for which day of the week the dosage formulations contained therein are to be taken by the user, and optionally where the drug delivery device or package, blister pack, clamshell or tray has one row for morning, breakfast or AM administration, and one row marked for evening, dinnertime or PM administration, each column or day will have two compartments, optionally where the compartment for morning, breakfast or AM administration is above the compartment for evening, dinnertime or PM administration,
and optionally each compartment has a foil or equivalent backing, or each compartment is an environmentally- (optionally moisture-, pathogen-, and/or light-) protected or sealed storage unit, and optionally the foil backing requires minimal finger strength to remove a dosage formulation (optionally one, two or three or more capsules, tablets, pills, geltabs or equivalents) in the compartment,
and optionally the blister package is a face seal blister package, a gang run blister package, a mock blister package, an interactive blister package or a slide blister package,
and optionally the drug delivery device or package, blister package, clamshell or tray is joined with board material which allows the product to be packaged, handled, hung, displayed and/or shipped without damaging the blister protection or seal, and optionally also provided with child resistant features,
and optionally the drug delivery device or package, blister package, clamshell or tray comprises a medical electronic monitory system that records administration time and transmits information to near-field communication (NFC) enabled mobile phone,
wherein optionally the kit is a travel kit comprising instructions of use by the traveler, wherein the instructions optionally comprise instructing the traveler to immediately take an indicated dosage, optionally a first arrange row of drugs on the drug delivery device or package, blister package, clamshell or tray, if they believe they have been in contact with or in near contact with or exposed to an individual that is infect or might be infected with coronavirus (e.g., a COVID-19) or to an individual having fever, sore throat, rigors or shakes, chest pain on breathing, coughing, diarrhea and/or muscle aches.

6. A method for treating, preventing, ameliorating, slowing the progress of, decreasing the severity of or preventing a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales, comprising administered to an individual in need thereof a therapeutic combination of drugs or drug, a pharmaceutical dosage form, a drug delivery device, or a product of manufacture of any of the preceding claims.

7. The method of chain 6, wherein the administered therapeutic combination of drugs or drug, pharmaceutical dosage form, drug delivery device, or product of manufacture comprises, or the method of administration comprises:
(a) lopinavir, ritonavir and oseltamivir (e.g., TAMIFLU™);
(b) lopinavir combined (formulated) with ritonavir, or KALETRA™, ALTERA™, ALUVIA™, KALMELTREX, LOPIMUNE™ or LOPINAVIR™, or lopinavir and ritonavir separately formulated;
(c) lopinavir combined (formulated) with ritonavir, or KALETRA™, ALTERA™, ALUVIA™, KALMELTREX, LOPIMUNE™ or LOPINAVIR™, and oseltamivir (e.g., TAMIFLU™), optionally also with inhaled chloroquine (or ARALEN™), chloroquine phosphate, chloroquine diphosphate and/or hydroxychloroquine (e.g., PLAQUENIL™) and/or oral chloroquine (or ARALEN™), chloroquine phosphate, chloroquine diphosphate and/or hydroxychloroquine (e.g., PLAQUENIL ™) simultaneously,
wherein optionally the dosage administration comprises: lopinavir about 200 mg twice daily, ritonavir about 50 mg twice daily, chloroquine about 250 mg twice daily, oseltamivir (TAMIFLU™) about 75 mg twice daily;
(d) lopinavir, ritonavir and oseltamivir (or TAMIFLU™); with inhaled chloroquine (or ARALEN™), chloroquine phosphate, chloroquine diphosphate and/or hydroxychloroquine (e.g., PLAQUENIL™) and/or oral chloroquine (or ARALEN™), chloroquine phosphate, chloroquine diphosphate and/or hydroxychloroquine (e.g., PLAQUENIL ™) simultaneously;
(e) lopinavir and oseltamivir (e.g., TAMIFLU™);
(f) ritonavir and oseltamivir (e.g., TAMIFLU™);
(g) remdesivir (e.g., GS-5734™, Gilead Sciences) alone, or oseltamivir (e.g., TAMIFLU™) and remdesivir (e.g., GS-5734™, Gilead Sciences);
(h) oseltamivir (e.g., TAMIFLU™) and efavirenz (e.g., SUSTIVA™);
(i) oseltamivir (e.g., TAMIFLU™) and nevirapine (or the combination efavirenz with emtricitabine and tenofovir, or ATRIPLA™);
(j) oseltamivir (or TAMIFLU™) and amprenavir (e.g., AGENERASE™);
(k) oseltamivir (e.g., TAMIFLU™) and nelfinavir (e.g., VIRACEPT™); or
(l) a thiazolide class drug, optionally nitazoxanide (or ALINIA™, NIZONIDE™) or tizoxanide (or 2-Hydroxy-N-(5-nitro-2-thiazolyl)benzamide), with or in combination with any of (a) to (i), or the thiazolide class drug (e.g., nitazoxanide or tizoxanide) and oseltamivir (or TAMIFLU™);
(m) plitidepsin (also known as dehydrodidemnin B), or APLIDIN™ (PharmaMar, S.A.);
(n) an inhibitor or S-phase kinase-associated protein 2(SKP 2 ), or dioscin, or niclosamide, or NICLOCIDE™, FENASAL™, or PHENASAL™;
(o) ribavirin, interferon beta 1b, or interferon alfa-2b,, or a combination of an interferon alpha or beta, or a combination of ribavirin and interferon beta, or a combination of lopinavir and ritonavir and interferon-beta-1b or interferon alfa-2b;
(p) abacavir, actemra, acyclovir, e.g., (ACICLOVIR™), adefovir, amantadine, ampligen, amprenavir (e.g., AGENERASE™), aprepitant, arbidol, atazanavir, atripla, balavir, baloxavir marboxil (XOFLUZA™), bepotastine, bevirimat, bictegravir, biktarvy, brilacidin, cidofovir, caspofungin, combivir, cobicstat, colisitin, cocaine, darunavir, delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, favilavir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II and/or interferon type III), lamivudine, lopinavir, loviride, ledipasvir, leronlimab, maraviroc, methisazone, moroxydine, nelfinavir, nevirapine, nexavir, nitazoxanide, norvir, a nucleoside analogue (optionally didanosine, vidarabine, galidesivir (e.g., BCX4430, IMMUCILLIN-A™), remdesivir (e.g., GS-5734™, Gilead Sciences), cytarabine, gemcitabine, emtricitabine, lamivudine, zalcitabine, abacavir, acyclovir, entecavir, stavudine, telbivudine, zidovudine, idoxuridine and/or trifluridine or any combination thereof), oseltamivir (or TAMIFLU™), peginterferon alfa-2a, penciclovir, peramivir (e.g., RAPIVAB™), perfenazine, pleconaril, plurifloxacin, podophyllotoxin, pyramidine, raltegravir, remdesivir, rifampicin, ribavirin, rilpivirine, rimantadine, ritonavir, saquinavir, sofosbuvir, stavudine, telaprevir, tegobuv, tenofovir alafenamide, tenofovir disoproxil, tenofovir, tipranavir, trifluridine, abacavir, lamivudine and zidovudine (e.g. TRIZIVIR™), tromantadine, emtricitabine and tenofovir (e.g., TRUVADA™), valaciclovir (e.g., VALTREX™), valganciclovir, valrubicin, vapreotide, vicriviroc, vidarabine, viramidine, velpatasvir, vivecon, zalcitabine, zanamivir (e.g., RELENZA™), zidovudine or any combination thereof;
(q) chloroquine (or ARALEN™), chloroquine phosphate, chloroquine diphosphate and/or hydroxychloroquine (e.g., PLAQUENIL™) with or in combination with any of (a) to (p), or chloroquine chloroquine phosphate, chloroquine diphosphate and/or hydroxychloroquine (e.g., PLAQUENIL™) and oseltamivir (or TAMIFLU™);
(r) an mucolytic therapy or drug, optionally acetylcysteine, ambroxol, bromhexine, carbocisteine, erdosteine, mecysteine or domase alfa, or an expectorant, optionally guaifenesin;
(s) a viral, or a coronavirus or a COVID-19, protease inhibitor, optionally ASC09 (CAS registry no. 1000287-05-7) (Janssen Research and Development, LLC), ritonavir or ASC09 and ritonavir, or a JAK1/2 inhibitor (optionally baricitinib), optionally compound 11r (University of Lubeck, Germany, see e.g., Zhang et al J. Med Chem 2020, Feb. 11, 2020), or darunavir, cobicistat or darunavir and cobicistat;
(t) an angiotensin-converting enzyme 2(ACE 2 ) inhibitor, optionally to block the site of viral spike protein interaction for anti-SARS-CoV-2 infection control;
(u) an anti-vascular endothelial growth factor (VEGF) (optionally VEGF-A) drug or antibody, optionally bevacizumab;
(v) a serine protease inhibitor, optionally camostat;
(w) anti-PD-1 checkpoint inhibitor, optionally camrelizumab;
(x) a compound or antibody capable of binding complement factor CS and blocking membrane attack complex formation, optionally eculizumab;
(y) a cathepsin inhibitor, optionally a cathepsin K, B or L inhibitor, optionally relacatib;
(z) thalidomide, or thalidomide and glucocorticoid (optionally low-dose glucocorticoid), or and thalidomide and celecoxib;
(aa) an antibacterial antibiotic or a macrolide drug, wherein optionally the macrolide drug comprises azithromycin (e.g., ZITHROMAX™, or AZITHROCIN™), clarithromycin (e.g., BIAXIN™), erythromycin (e.g., ERYTHROCIN™), or fidaxomicin (e.g., DIFICID™ or DIFICLIR™), troleandomycin (e.g., TEKMISIN™), tylosin (e.g., TYLOCINE™ or TYLAN™), solithromycin (e.g., SOLITHERA™), oleandomycin (or SIGMAMYCINE™), midecamycin, roxithromycin, kitasamycin or turimycin, josamycin, carbomycin or magnamycin, and/or spiramycin;
(bb) chloroquine (or ARALEN™), chloroquine phosphate, chloroquine diphosphate and/or hydroxychloroquine (e.g., PLAQUENIL™) with (or formulated with) or in combination with any of (a) to (y), or chloroquine, chloroquine phosphate, chloroquine diphosphate and/or hydroxychloroquine (e.g., PLAQUENIL™) and oseltamivir (or TAMIFLU™);
(cc) chloroquine (e.g., ARALEN™), chloroquine phosphate, chloroquine diphosphate and/or hydroxychloroquine (e.g., PLAQUENIL™);
(dd) any one or several or all of (a) to (r), or any therapeutic combination of drugs or drug, pharmaceutical dosage form of any of the preceding claims, are administered as an inhaled aerosol, powder or liquid or other inhalation formulation, or are administered with inhaled (e.g., aerosol or power administered by inhaler) chloroquine (or ARALEN™), chloroquine phosphate, chloroquine diphosphate and/or hydroxychloroquine (e.g., PLAQUENIL™),
and/or the any one or several or all of (a) to (r), or any therapeutic combination of drugs or drug, pharmaceutical dosage form of any of the preceding claims, are administered orally, intramuscularly, subcutaneously, topically, by enema, intravaginally, or intravenously,
and optionally are administered with oral (e.g., pills, tablets, capsules, liquids) chloroquine (or ARALEN™), chloroquine phosphate, chloroquine diphosphate and/or hydroxychloroquine (e.g., PLAQUENIL™),
wherein the inhaled (e.g., aerosol or power administered by inhaler) therapeutic combination of drugs or drug, pharmaceutical dosage form, drug delivery device, or product of manufacture is or are administered simultaneously or overlapping or sequentially; or
(ee) any combination of (a) to(s) simultaneously administered in both inhaled (e.g., aerosol or power administered by inhaler) and oral (e.g., pills, tablets, capsules, liquids) forms; or
(ff) any combination of (a) to (ee).

8. The method of claim 6, wherein the lopinavir, ritonavir and oseltamivir are formulated or packaged for administration as or dosing in the ratio of 25:100:75 of lopinavir: ritonavir: oseltamivir.

9. The method of claim 6, wherein the chloroquine (e.g., ARALEN™), chloroquine phosphate, chloroquine diphosphate, hydroxychloroquine (e.g., PLAQUENIL™), lopinavir, ritonavir and/or oseltamivir, and/or the anti-viral drug or medication, or anti-microbial drug, are administered enterally or parenterally, or are administered orally, intramuscularly (IM), intravenously (IV), by inhalation, or by inhalation when formulated as an aerosol, a spray, a microparticle, a nanoparticle, or a powder.

10. The method of claim 6, wherein the chloroquine (e.g., ARALEN™), chloroquine phosphate, chloroquine diphosphate, hydroxychloroquine (e.g., PLAQUENIL™), lopinavir, ritonavir and/or oseltamivir, and/or the anti-viral drug or medication, or anti-microbial drug, are administered one to ten times per day (e.g., bid, tid, or 5, 6, 7, 8, 9, or 10 or more times a day) depending on the stage of the condition (for example, exposed to infection, positivity in blood but asymptomatic, or symptomatic, mild or severe.

11. The method of claim 6, comprising first administering (alone) for about 7 days (or 2, 3, 4, 5, 6 or 7 or more days) oseltamivir (or TAMIFLU™) at 3 times per day (tid) (or alternatively twice a day (bid) or four times a day or more), followed by a blood sample to be taken for virus testing; and if and when virus blood positivity is confirmed, or viral infection is otherwise confirmed, the osteltamivir is supplemented by the lopinavir and ritonavir (which can be administered together or separately) three times daily (tid) (or alternatively twice a day (bid) or four times a day or more), while continuing the osteltamivir, for example, all three medications three (or two or four or more) times daily or 9 (or more) medications daily.

12. The method of claim 6, wherein the duration of the combined drug therapy, or the chloroquine (e.g., ARALEN™), chloroquine phosphate, chloroquine diphosphate or hydroxychloroquine (e.g., PLAQUENIL™) therapy, is about 2, 3, 4, 5, 6, 7, 8, 9, 10, 11, 12, 13, 14, 15, 16, 17, 18, 19, 20 or 21 or more or more days or for as long as the patient tests positive for the coronavirus (e.g., a COVID-19) infection, or for at least 2, 3, 4, 5, 6, 7, 8, 9, 10, 11, 12, 13, 14, 15, 16, 17, 18, 19, 20 or 21 or more or more days after the patient no longer tests positive for coronavirus (e.g., a COVID-19), or wherein the combined drug therapy, or
the chloroquine (e.g., ARALEN™), chloroquine phosphate, chloroquine diphosphate or hydroxychloroquine (e.g., PLAQUENIL™) therapy, begins (commences) as soon as an individual (a patient) learns (understands) he or she was in close proximity to a coronavirus (e.g., a COVID-19) positive individual, or believes that he or she may have been in close proximity to a coronavirus (e.g., a COVID-19) positive individual, or begins (commences) as soon as the individual (the patient) test positive for coronavirus (e.g., a COVID-19), or
the chloroquine (e.g., ARALEN™), chloroquine phosphate, chloroquine diphosphate or hydroxychloroquine (e.g., PLAQUENIL™) therapy, continues until at least one, or at least two, tests show the individual (the patient) is negative for coronavirus (e.g., a COVID-19), or continues until all body fluids or samples (optionally respiratory or oral swabs or fluids, sputum, serology or blood or serum, and stool or fecal samples) test negative for coronavirus (e.g., a COVID-19),
and optionally the individual (the patient) is retested at least every week, biweek or month to test or reinfection or reemergence or reoccurrence of viral infection or activity, and if the individual (the patient) again tests positive, then reinitiate treatment as used when the individual was first diagnosed with coronavirus (e.g., a COVID-19) infection.

13. The method of claim 6, wherein the combined drug therapy, or the chloroquine (e.g., ARALEN™), chloroquine phosphate, chloroquine diphosphate or hydroxychloroquine (e.g., PLAQUENIL™) therapy, comprises administering the chloroquine (e.g., ARALEN™), chloroquine phosphate, chloroquine diphosphate or hydroxychloroquine (e.g., PLAQUENIL™):
(a) until, or increasing the dosage of the chloroquine (e.g., ARALEN™), chloroquine phosphate, chloroquine diphosphate or hydroxychloroquine (e.g., PLAQUENIL™), until a steady state chloroquine (e.g., ARALEN™), chloroquine phosphate, chloroquine diphosphate or hydroxychloroquine (e.g., PLAQUENIL™) plasma concentration of at least 1 μg/mL (or at least approximately 3.125μM/L) is achieved, or until a whole blood concentration of at least about 16 μM/L is achieved, and optionally sustained, until the patient's viremia becomes undetectable; or
(b) intravenously (IV) at a dosage of between about 10 mg/kg to 20 mg/kg, or at about 15 mg/kg, over a one to 6 hour period, or over four hour period, and optionally the IV infusion is repeated, optionally every 6 to 12 hours, optionally for between about 2 to 20 repeat infusions.

14. The method of claim 6, wherein the combined drug therapy, or the chloroquine (e.g., ARALEN™), chloroquine phosphate, chloroquine diphosphate or hydroxychloroquine (e.g., PLAQUENIL™) therapy, is formulated with or is administered before, during or after: an anti-coronavirus vaccine or a vaccination with an anti-coronavirus (e.g., an anti-COVID-19) vaccine, or administration of a passive immunity therapy (e.g., infusion (e.g., intravenous infusion, e.g., infusion of a hyperimmune globulin) of one or more antibodies capable of specifically binding to or neutralizing or stopping the multiplication or spread of a coronavirus (e.g., a COVID-19).

15. The method of claim 6, wherein the combined drug therapy, or the chloroquine (e.g., ARALEN™), chloroquine phosphate, chloroquine diphosphate or hydroxychloroquine (e.g., PLAQUENIL™) therapy, is administered with (optionally before, during and/or after), or formulated with, an anti-inflammatory therapy or at least one anti-inflammatory therapy drug, wherein optionally the anti-inflammatory therapy or drug comprises: a sphingosine kinase-2 (SK2) selective inhibitor (e.g., opaganib (e.g., YELIVA™), sirolimus, a JAK1/2/TYK2 inhibitor (optionally ruxolitinib), an anti-CD47 mAb (optionally meplazumab), COX (e.g., COX2) inhibitor, a glucocorticoid and/or an immunosuppressive agent, and optionally the immunosuppressive agent comprises tocilizumab or fingolimod.

16. The method of claim 6, wherein the combined drug therapy, or the chloroquine (e.g., ARALEN™), chloroquine phosphate, chloroquine diphosphate or hydroxychloroquine (e.g., PLAQUENIL™) therapy, is formulated with or is administered with (optionally before, during and/or after) administration of:
(a) an mucolytic therapy or drug, optionally acetylcysteine, ambroxol, bromhexine, carbocisteine, erdosteine, mecysteine or domase alfa, or an expectorant, optionally guaifenesin;
(b) a viral, or a coronavirus or a COVID-19, protease inhibitor, optionally ASC09 (CAS registry no. 1000287-05-7) (Janssen Research and Development, LLC), ritonavir or ASC09 and ritonavir, or a JAK1/2 inhibitor (optionally baricitinib), optionally compound 11r (University of Lubeck, Germany, see e.g., Zhang et al J. Med Chem 2020, Feb. 11, 2020), or darunavir, cobicistat or darunavir and cobicistat;
(c) an angiotensin-converting enzyme 2(ACE 2 ) inhibitor, optionally to block the site of viral spike protein interaction for anti-SARS-CoV-2 infection control;
(d) an anti-vascular endothelial growth factor (VEGF) (optionally VEGF-A) drug or antibody, optionally bevacizumab;
(e) a serine protease inhibitor, optionally camostat;
(f) anti-PD-1 checkpoint inhibitor, optionally camrelizumab;
(g) a compound or antibody capable of binding complement factor C5 and blocking membrane attack complex formation, optionally eculizumab;
(h) a cathepsin inhibitor, optionally a cathepsin K, B or L inhibitor, optionally relacatib;
(i) thalidomide, or thalidomide and glucocorticoid (optionally low-dose glucocorticoid), or and thalidomide and celecoxib, or
(j) any combination of (a) to (i).
